(19) United States
(12) Patent Application Publication
(10) Pub. No.: US 2025/0009213 A1
(43) Pub. Date: Jan. 9, 2025

(54) ENDOSCOPE SYSTEM, PROCEDURE SUPPORT METHOD, AND RECORDING MEDIUM

(71) Applicants: OLYMPUS CORPORATION, Tokyo (JP); National Cancer Center, Tokyo (JP)

(72) Inventors: Naoya HATAKEYAMA, Tokyo (JP); Masafumi HARAGUCHI, Tokyo (JP); Masaaki ITO, Tokyo (JP); Shigehiro KOJIMA, Tokyo (JP); Daichi KITAGUCHI, Tokyo (JP); Hiro HASEGAWA, Tokyo (JP); Yuki FURUSAWA, Tokyo (JP)

(73) Assignees: OLYMPUS CORPORATION, Tokyo (JP); National Cancer Center, Tokyo (JP)

(21) Appl. No.: 18/897,470

(22) Filed: Sep. 26, 2024

Related U.S. Application Data
(63) Continuation of application No. PCT/JP2023/010648, filed on Mar. 17, 2023.
(60) Provisional application No. 63/327,416, filed on Apr. 5, 2022.

(51) Int. Cl. A61B 1/045; A61B 1/00

(52) U.S. Cl. CPC A61B 1/045; A61B 1/00006; A61B 1/00009; A61B 1/0002; A61B 1/0004; A61B 1/00149; A61B 1/00183

(57) ABSTRACT

An endoscope system includes: an endoscope including an elongated portion in which an imaging optical system is arranged; an arm supporting the endoscope; and a unit capable of changing a field-of-view direction of the endoscope, wherein each library data stored in a storage includes a parameter related to relative position and attitude between the imaging optical system and an observation object, the parameter includes a first parameter or a second parameter, the first parameter determined by a sum of an inclination of a longitudinal axis of the elongated portion and an amount of curvature of the changing unit on base coordinates, the first parameter related to an orientation of the imaging optical system on the base coordinates, the second parameter indicating an orientation of the imaging optical system as viewed from a coordinate of the observation object, and the unit or the arm based on the library data is controlled.

### CROSS-REFERENCE TO RELATED APPLICATIONS

This is a continuation of International Application PCT/JP2023/010648, with an international filing date of Mar. 17, 2023, which is hereby incorporated by reference herein in its entirety.

This application claims the benefit of U.S. Provisional Application No. 63/327,416, filed Apr. 5, 2022, which is hereby incorporated by reference herein in its entirety.

### TECHNICAL FIELD

The present disclosure relates to an endoscope system, a procedure support method, and a recording medium.

### BACKGROUND

Conventionally, techniques for reducing an operator's effort to perform manipulations related to procedures are known (for example, refer to PTL 1 and PTL 2). The technique described in PTL 1 restores an endoscope to an original position and an original attitude by recording angle information of joints of a holder in advance and time-sequentially reproducing in reverse, angles of the joints of the holder based on the angle information. The technique described in PTL 2 controls a function of an energy treatment tool or the like during a surgical procedure based on a machine learning model having been trained using surgical tool image data or images of anatomical structures including a procedural state and a procedural type of the surgical procedure.

### SUMMARY

A first aspect of the present disclosure is an endoscope system comprising: an endoscope that comprises an imaging optical system for photographing an observation object; an electric arm that changes a position and an attitude of the endoscope while supporting the endoscope; a field-of-view direction changing unit capable of changing a field-of-view direction of the endoscope; a storage apparatus that stores a plurality of pieces of library data for realizing an endoscopic field of view, each piece of library data being associated with each procedural scene; and at least one processor, wherein: the endoscope comprises an elongated portion in which the imaging optical system is arranged at a tip portion thereof, each piece of library data comprises at least one relative parameter related to a relative position and a relative attitude between the imaging optical system and the observation object, the relative parameter comprises at least one of a first parameter or a second parameter, the first parameter being determined by a sum of an inclination of a longitudinal axis of the elongated portion and an amount of curvature of the field-of-view direction changing unit on base coordinates, the first parameter being related to an orientation of the imaging optical system on the base coordinates, the second parameter indicating an orientation of the imaging optical system as viewed from a coordinate of the observation object, and the processor is configured to call the library data associated with any procedural scene from the storage apparatus, and subsequently control at least one of the field-of-view direction changing unit or the electric arm based on the called library data.

A second aspect of the present disclosure is a procedure support method comprising steps of: calling, from a storage apparatus, library data associated with any procedural scene among a plurality of pieces of library data for realizing an endoscopic field of view, each piece of library data being associated with each procedural scene; and changing at least any one of a field-of-view direction, a position, or an attitude of an endoscope for photographing an observation object based on the called library data, wherein: the endoscope comprises an elongated portion in which the imaging optical system is arranged at a tip portion thereof, each piece of the library data comprises at least one relative parameter related to a relative position and a relative attitude between an imaging optical system of the endoscope and the observation object, and the relative parameter comprises at least one of a first parameter or a second parameter, the first parameter being determined by a sum of an inclination of a longitudinal axis of the elongated portion and an amount of curvature of the field-of-view direction changing unit on base coordinates, the first parameter being related to an orientation of the imaging optical system on the base coordinates, the second parameter indicating the orientation of the imaging optical system as viewed from a coordinate of the observation object.

A third aspect of the present disclosure is a non-transitory computer-readable recording medium storing a procedure support program causing a computer to execute steps of: calling, from a storage apparatus, library data associated with any procedural scene among a plurality of pieces of library data for realizing an endoscopic field of view, each piece of library data being associated with each procedural scene; and changing at least any one of a field-of-view direction, a position, or an attitude of an endoscope for photographing an observation object based on the called library data, wherein: the endoscope comprises an elongated portion in which the imaging optical system is arranged at a tip portion thereof, each piece of the library data comprises at least one relative parameter related to a relative position and a relative attitude between an imaging optical system of the endoscope and the observation object, and the relative parameter comprises at least one of a first parameter or a second parameter, the first parameter being determined by a sum of an inclination of a longitudinal axis of the elongated portion and an amount of curvature of the field-of-view direction changing unit on base coordinates, the first parameter being related to an orientation of the imaging optical system on the base coordinates, the second parameter indicating the orientation of the imaging optical system as viewed from a coordinate of the observation object.

### BRIEF DESCRIPTION OF DRAWINGS

FIG. 1 is a schematic configuration diagram of an endoscope system according to a first embodiment of the present disclosure.

FIG. 2 is a diagram for describing an example of a schematic configuration and relative parameters of an electric scope.

FIG. 3 is a diagram for describing a control method by a control apparatus.

FIG. 4 is a flow chart for describing a procedure support method and a procedure support program according to the first embodiment of the present disclosure.

FIG. 5 is a diagram for describing a relationship between an inclination of the electric scope and an orientation of a camera.

FIG. 6 is a diagram for describing an orientation of an imaging optical system as viewed from base coordinates.

FIG. 7 is a diagram for describing a relationship between an attitude of an observation object and an orientation of the camera.

FIG. 8 is a diagram for describing an orientation of the imaging optical system with respect to the observation object.

FIG. 9 is a diagram for describing a relationship between a direction in which an elongated observation object runs and an inclination around an optical axis of the camera.

FIG. 10 is a diagram showing an example of an endoscopic field of view in a state where the direction in which the elongated observation object runs faces a lateral direction with respect to a screen of a monitor.

FIG. 11 is a diagram for describing a method of adjusting a roll angle of the electric scope in accordance with a direction in which the elongated observation object runs.

FIG. 12 is a diagram for describing a method of adjusting the roll angle of the electric scope in accordance with an attitude of a patient or an operating table.

FIG. 13A is a diagram showing a relationship between coordinates of the operating table or the patient and base coordinates during normal time.

FIG. 13B is a diagram showing a relationship between coordinates of an inclined operating table or an inclined patient and uncalibrated base coordinates.

FIG. 13C is a diagram showing a relationship between the coordinates of the inclined operating table or the inclined patient and calibrated base coordinates.

FIG. 14A is a diagram showing a relationship between coordinates of the operating table or the patient and base coordinates during normal time.

FIG. 14B is a diagram showing a relationship between coordinates of the inclined operating table or the inclined patient and an orientation of the electric scope when library data is not calibrated.

FIG. 14C is a diagram showing a relationship between coordinates of the inclined operating table or the inclined patient and the orientation of the electric scope when library data is calibrated.

FIG. 15 is a diagram for describing that a main controller switches between pieces of library data according to a determination of a procedural scene by an AI.

FIG. 16 is a diagram for describing that the main controller switches between pieces of library data according to a specification of a procedural scene by an operator.

FIG. 17 is a diagram for describing that the main controller switches between pieces of library data according to an instruction issued by the operator.

FIG. 18 is a diagram for describing that the main controller switches between pieces of library data according to a treatment position in the patient.

FIG. 19 is a diagram for describing a treatment position with an insertion point in the patient as an origin.

FIG. 20 is a diagram for describing another treatment position with an insertion point in the patient as an origin.

FIG. 21 is a diagram for describing a control method of the endoscope system when adopting a roll angle around a visual axis as library data.

FIG. 22 is a diagram for describing the control method of the endoscope system when creating a downward endoscopic field of view.

FIG. 23 is a schematic configuration diagram of an oblique-viewing endoscope of an endoscope system according to a second embodiment of the present disclosure.

FIG. 24 is a diagram for describing a control method by a control apparatus.

FIG. 25 is a diagram for describing that a field-of-view direction of the oblique-viewing endoscope changes around a longitudinal axis of a lens barrel unit.

FIG. 26 is a diagram for describing a look-up field of view of the oblique-viewing endoscope.

FIG. 27 is a diagram for describing a field of view when a curved part of an electric scope is curved by around 30 degrees in an upward direction.

FIG. 28 is a diagram for describing a look-down field of view of the oblique-viewing endoscope.

FIG. 29 is a diagram for describing a field of view when the curved part of the electric scope is curved by around 30 degrees in a downward direction.

FIG. 30 is a diagram for describing that a visual axis of a camera rotates around an axial line.

FIG. 31 is a schematic configuration diagram of a forward-viewing endoscope of an endoscope system according to a third embodiment of the present disclosure.

FIG. 32 is a diagram for describing a control method by a control apparatus.

FIG. 33 is a diagram for describing that a visual axis of a camera rotates around an axial line.

FIG. 34 is a diagram showing a situation where an observation object is captured at a center of an endoscopic field of view.

FIG. 35 is a diagram showing a situation where an observation object is positioned at a center of a screen.

FIG. 36 is a diagram showing a situation where an observation object is captured at an end of an angle of view.

FIG. 37 is a diagram showing a situation where a location previously hidden from view becomes visible due to an endoscopic field of view of an angle of looking into an observation object.

### DESCRIPTION OF EMBODIMENTS

### First Embodiment

An endoscope system, a procedure support method, and a procedure support program according to a first embodiment of the present disclosure will be hereinafter described with reference to the drawings.

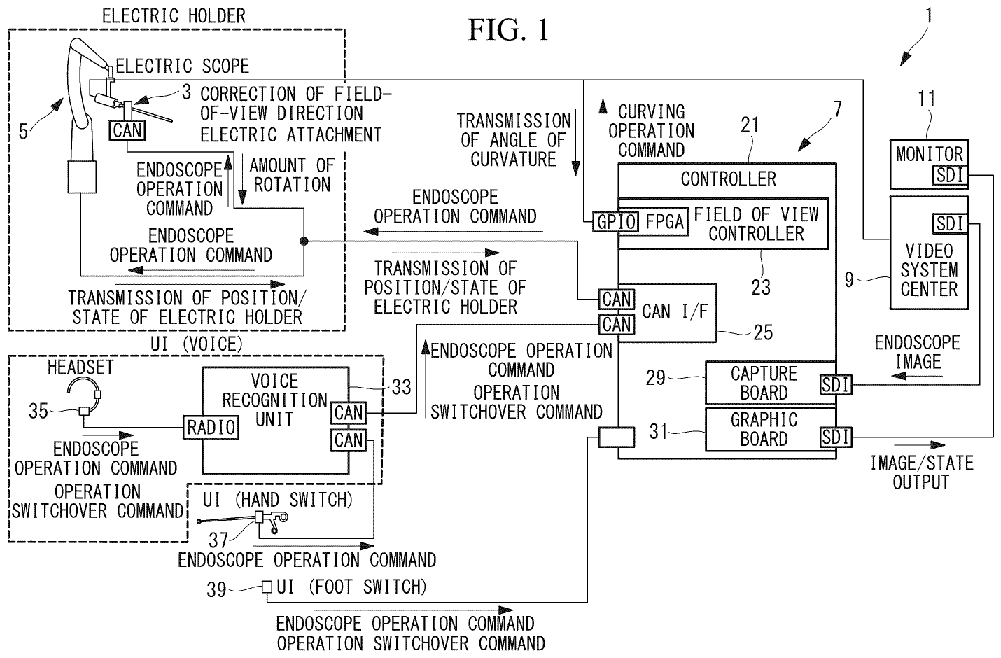

As shown in FIG. 1, an endoscope system 1 according to the present embodiment includes an electric scope (endoscope) 3 which acquires an image of the inside of a body cavity of a patient, a robot arm (electric arm) 5 which supports the electric scope 3, and a control apparatus 7 which controls the electric scope 3 and the robot arm 5. In FIG. 1, reference sign 9 denotes a video system center and reference sign 11 denotes a monitor. The video system center 9 is connected to the electric scope 3 and the control apparatus 7. The monitor 11 is connected to the control apparatus 7.

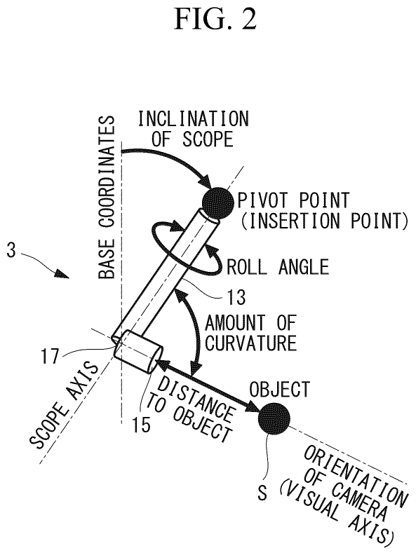

As shown in FIG. 2, the electric scope 3 includes an elongated insertion portion (elongated portion) 13 to be inserted into a body cavity of the patient, a camera (imaging optical system) 15 provided at a tip portion of the insertion portion 13, a curved part (field-of-view direction changing unit) 17 which causes an inclination angle of an endoscopic field of view of the camera 15 relative to a longitudinal axis of the insertion portion 13 to change, and a curved motor which drives the curved part 17 and a roll motor which rolls and rotates the insertion portion 13 around the longitudinal axis (both motors not illustrated).

For example, the camera 15 is constituted of at least one lens and an imager such as a CCD (Charge Coupled Device) or a CMOS (Complementary Metal-Oxide-Semiconductor). The camera 15 may be a monocular camera or a stereo camera.

Furthermore, the electric scope 3 has a ranging function of measuring a distance to an observation object captured in a field of view of the camera 15. Known mechanisms can be adopted as a mechanism of realizing the ranging function.

The robot arm 5 is, for example, an electric holder of a general-purpose 6-axis articulated robot which movably holds the electric scope 3 at any position. The robot arm 5 includes, for each joint, a motor (not illustrated) which operates each joint.

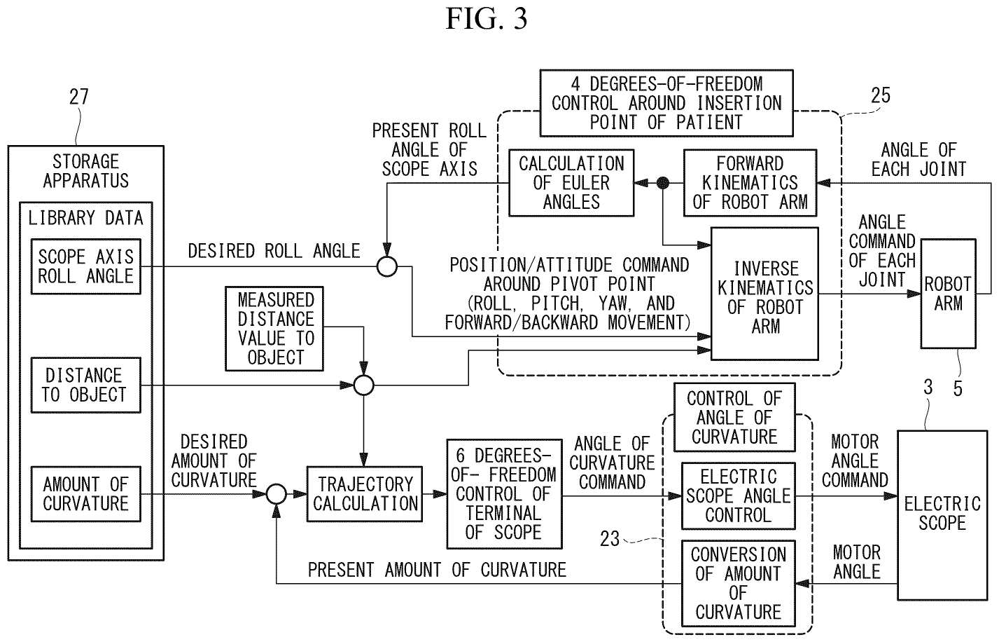

The control apparatus 7 is realized by, for example, a dedicated computer or a general-purpose computer. In other words, as shown in FIGS. 1 and 3, the control apparatus 7 includes a main controller (processor) 21 which is a CPU (Central Processing Unit) or a GPU (Graphics Processing Unit), a field of view controller (processor) 23 and a position/attitude controller (processor) 25, a main storage apparatus (not illustrated) such as a RAM (Random Access Memory) to be used as a working area of the respective controllers 21, 23, and 25, and an auxiliary storage apparatus (storage apparatus) 27.

The auxiliary storage apparatus 27 is a computer-readable non-transitory recording medium such as an SSD (Solid State Drive) or an HDD (Hard Disk Drive). The auxiliary storage apparatus 27 stores a procedure support program which causes each of the controllers 21, 23, and 25 to execute processing and a plurality of pieces of library data for realizing an endoscopic field of view of the electric scope 3, each of the pieces of library data being associated with each procedural scene. The main storage apparatus and the auxiliary storage apparatus 27 may be configured to be connected to the control apparatus 7 via a network.

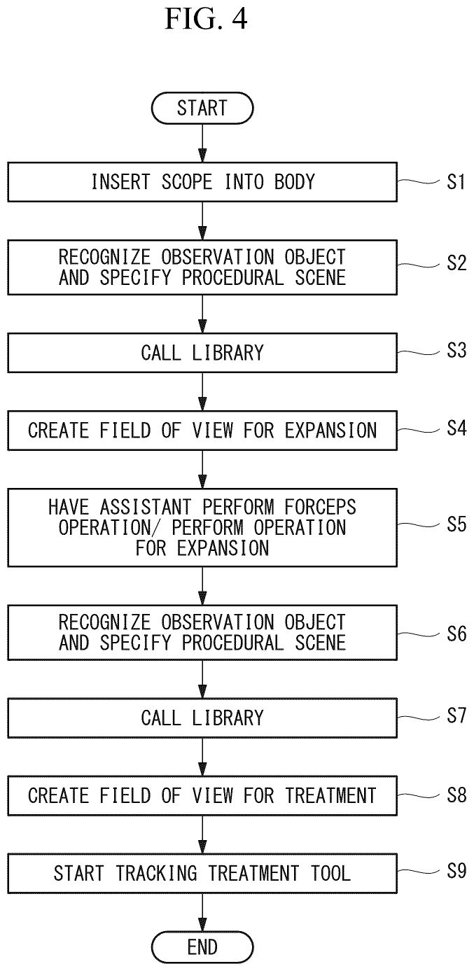

For example, as shown in FIG. 4, the procedure support program causes the respective controllers 21, 23, and 25 of the control apparatus 7 to execute: a step of recognizing an observation object S photographed by the camera 15 and a step of specifying a procedural scene by processing image information acquired by the electric scope 3 (steps S2 and S6); steps of calling library data associated with the specified procedural scene from the auxiliary storage apparatus 27 (steps S3 and S4); and steps of changing at least one of an angle of the camera 15 and a position or an attitude of the electric scope 3 based on the called library data (steps S4 and S8).

The main controller 21 includes a capture board 29 which captures an endoscopic image from the video system center 9 and a graphic board 31 which outputs the endoscopic image and a state signal. By processing an endoscopic image, the main controller 21 recognizes the observation object S on the endoscopic image and specifies a procedural scene in the endoscopic image. In addition, the main controller 21 calls library data associated with the specified procedural scene from the auxiliary storage apparatus 27.

The field of view controller 23 is connected to the electric scope 3 and sends a curving operation command to the electric scope 3 and receives angle of curvature information from the electric scope 3.

The position/attitude controller 25 is connected to the electric scope 3 and the robot arm 5 and to a voice recognition unit 33. The position/attitude controller 25 sends an endoscope operation command to the electric scope 3 and receives an input of an amount of rotation around the longitudinal axis of the insertion portion 13 from the electric scope 3. In addition, the position/attitude controller 25 sends the endoscope operation command to the robot arm 5 and receives a signal of a position and a state (attitude) of the robot arm 5 from the robot arm 5.

In addition, a headset (input unit) 35, a hand switch (input unit) 37, a foot switch (input unit) 39, and the like are connected as various user interfaces (UIs) to the control apparatus 7. The headset 35 enables an operator to input an endoscope operation command, an operation switchover command, and the like by voice. The endoscope operation command, the operation switchover command, and the like inputted from the headset 35 are sent to the position/attitude controller 25 via the voice recognition unit 33.

For example, the hand switch 37 is mounted to a treatment tool and the operator can input the endoscope operation command by an operation at hand. The endoscope operation command inputted from the hand switch 37 is sent to the position/attitude controller 25 via the voice recognition unit 33. The foot switch 39 enables an operator to input the endoscope operation command and the operation switchover command by an operation using a foot. The endoscope operation command and the operation switchover command inputted from the foot switch 39 are sent to the main controller 21.

The plurality of pieces of library data stored in the auxiliary storage apparatus 27 include at least one relative parameter related to a relative position and a relative attitude of the electric scope 3 with respect to the camera 15 and the observation object S to be photographed by the camera 15. Examples of the relative parameter include scope-axis roll angle information, distance information on a distance between the camera 15 and the observation object S, and amount-of-curvature information on an amount of curvature of the curved part 17 of the electric scope 3.

As shown in FIG. 2, the scope-axis roll angle information represents a roll angle from an initial state around the longitudinal axis of the insertion portion 13 of the electric scope 3. The distance information on the distance between the camera 15 and the observation object S represents a distance between the camera 15 and the observation object S in a direction along a visual axis of the camera 15. The amount-of-curvature information on the amount of curvature of the curved part 17 represents an orientation of the camera 15 with respect to a scope axis of the electric scope 3. Hereinafter, these pieces of information will be referred to as roll angle information, distance information, and amount-of-curvature information. In other words, in the auxiliary storage apparatus 27, an endoscopic field of view suitable for each procedural scene has been set in advance for each procedural scene and at least any one of roll angle information, distance information, and amount-of-curvature information for realizing each endoscopic field of view is stored in association with each procedural scene.

After calling the roll angle information, the distance information and/or the amount-of-curvature information associated with any procedural scene from the auxiliary storage apparatus 27, the control apparatus 7 controls at least one of the curved part 17 of the electric scope 3 or the robot arm 5 based on each of the called pieces of information.

For example, when the control apparatus 7 calls the distance information from the auxiliary storage apparatus 27, the control apparatus 7 matches an actual distance between the camera 15 and the observation object S which is calculated from image information of the observation object S with the called distance information by controlling at least one of the curved part 17 of the electric scope 3 or the robot arm 5.

Specifically, as shown in FIG. 3, the main controller 21 calculates a trajectory for matching the distance between the camera 15 and the observation object S with the distance information by comparing a present measured distance value to the observation object S and the distance information called from the auxiliary storage apparatus 27. The present measured distance value to the observation object S is measured by the ranging function of the electric scope 3. In addition, after distribution to a joint control command of the robot arm 5 and an angle of curvature command of the electric scope 3, the main controller 21 inputs the joint control command to the position/attitude controller 25 and inputs the angle of curvature command to the field of view controller 23.

According to the joint control command, the position/attitude controller 25 uses inverse kinematics of the robot arm 5 to determine a drive amount of each joint required to match the distance between the camera 15 and the observation object S with the distance information. The determined drive amount of each joint is inputted to each motor of the robot arm 5 as an angle command for each joint (endoscope operation command).

On the other hand, according to the angle of curvature command, the field of view controller 23 determines an angle of curvature of the curved part 17 required to match the distance between the camera 15 and the observation object S with the distance information. The determined angle of curvature of the curved part 17 is inputted to the motor of the electric scope 3 as a motor angle command (curving operation command).

Due to each joint of the robot arm 5 moving according to each angle command and the curved part 17 of the electric scope 3 curving according to the motor angle command, an endoscopic field of view in which the distance between the camera 15 and the observation object S matches the distance information is obtained.

In addition, for example, when the control apparatus 7 calls the amount-of-curvature information from the auxiliary storage apparatus 27, the control apparatus 7 matches an orientation of the camera 15 relative to the observation object S which is calculated from image information of the observation object S with the called amount-of-curvature information by controlling at least one of the curved part 17 of the electric scope 3 or the robot arm 5.

Specifically, the main controller 21 calculates a trajectory for matching the amount of curvature of the curved part 17 with the amount-of-curvature information by comparing a present amount of curvature of the curved part 17 with the amount-of-curvature information called from the auxiliary storage apparatus 27. In addition, after distribution to a joint control command of the robot arm 5 and an angle of curvature command of the electric scope 3, the main controller 21 inputs the joint control command to the position/attitude controller 25 and inputs the angle of curvature command to the field of view controller 23.

According to the joint control command, the position/attitude controller 25 uses inverse kinematics of the robot arm 5 to determine a drive amount of each joint required to match the amount of curvature of the curved part 17 with the amount-of-curvature information. The determined drive amount of each joint is inputted to each motor of the robot arm 5 as an angle command for each joint (endoscope operation command).

On the other hand, according to the angle of curvature command, the field of view controller 23 determines an angle of curvature of the curved part 17 required to match the angle of curvature of the curved part 17 with the amount-of-curvature information. The determined angle of curvature of the curved part 17 is inputted to the motor of the electric scope 3 as a motor angle command (curving operation command).

Due to each joint of the robot arm 5 moving according to each angle command and the curved part 17 of the electric scope 3 curving according to the motor angle command, an endoscopic field of view in which the orientation of the camera 15 with respect to the observation object S matches the amount-of-curvature information is obtained.

In addition, for example, when the control apparatus 7 calls the roll angle information from the auxiliary storage apparatus 27, the control apparatus 7 matches an inclination of the observation object S around the optical axis of the camera 15 which is calculated from image information of the observation object S with the called roll angle information by controlling the robot arm 5.

Specifically, by comparing a present roll angle of the scope axis and the roll angle information, the main controller 21 determines variations of a position and an attitude around a pivot point required to match the roll angle of the scope axis with the roll angle information. The determined variation of the position and the attitude around the pivot point is inputted to the position/attitude controller 25 as a position/attitude command. For example, the position/attitude command includes a roll angle, a pitch angle, a yaw angle, and an amount of forward/backward movement of the electric scope 3.

According to the position/attitude command, the position/attitude controller 25 uses inverse kinematics of the robot arm 5 to determine a drive amount of each joint required to match the roll angle of the scope axis with the roll angle information. The determined drive amount of each joint is inputted to each motor of the robot arm 5 as an angle command for each joint (endoscope operation command). Due to each joint of the robot arm 5 moving according to each angle command, an endoscopic field of view in which an inclination of the observation object S around the optical axis of the camera 15 matches the roll angle information is obtained.

When controlling the robot arm 5, the position/attitude controller 25 calculates Euler angles (roll, pitch, and yaw) based on the angle of each joint using forward kinematics of the robot arm 5. Accordingly, the present roll angle of the scope axis is calculated. The calculated present roll angle of the scope axis is stored in the main controller 21.

When controlling the curved part 17 of the electric scope 3, the field of view controller 23 calculates a present amount of curvature of the curved part 17 by converting a motor angle of the curved part 17 into an amount of curvature. The calculated present amount of curvature is stored in the main controller 21.

Next, effects of the endoscope system 1, the procedure support method, and the procedure support program configured as described above will be described with reference to the flow chart in FIG. 4.

When supporting a procedure by an operator using the endoscope system 1, the procedure support method, and the procedure support program according to the present embodiment, first, the electric scope 3 is inserted into the body of a patient (step S1). In addition, due to the camera 15 of the electric scope 3 photographing the observation object S, image information is acquired. The acquired image information is inputted to the main controller 21 of the control apparatus 7 via the video system center 9.

Next, by processing the inputted image information, the main controller 21 recognizes the observation object S and specifies a procedural scene (step S2). In addition, after the main controller 21 calls library data associated with the specified procedural scene from the auxiliary storage apparatus 27 (step S3), the main controller 21 creates an endoscopic field of view for expansion (step S4). Expansion refers to an operation performed in order to incise tissue or make tissue more readily visible by applying tension to peripheral tissue by pulling the peripheral tissue with forceps handled by an assistant or the like.

For example, when the library data called from the auxiliary storage apparatus 27 is distance information, based on a present measured distance value between the camera 15 and the observation object S and the distance information, the main controller 21 determines a joint control command and an angle of curvature command for matching the distance between the camera 15 and the observation object S with the distance information.

Next, the position/attitude controller 25 determines a drive amount of each joint of the robot arm 5 based on the joint control command. In a similar manner, the field of view controller 23 determines an angle of curvature of the curved part 17 based on the angle of curvature command. In addition, due to each joint of the robot arm 5 and the curved part 17 of the electric scope 3 moving according to each command, an endoscopic field of view in which the distance between the camera 15 and the observation object S matches the distance information is created.

Furthermore, for example, when the library data called from the auxiliary storage apparatus 27 is amount-of-curvature information, based on a present amount of curvature of the curved part 17 and the amount-of-curvature information, the main controller 21 determines a joint control command and an angle of curvature command for matching the amount of curvature of the curved part 17 with the amount-of-curvature information.

Next, the position/attitude controller 25 determines a drive amount of each joint of the robot arm 5 based on the joint control command. In a similar manner, the field of view controller 23 determines the angle of curvature of the curved part 17 according to the angle of curvature command. In addition, due to each joint of the robot arm 5 and the curved part 17 of the electric scope 3 moving according to each command, an endoscopic field of view in which an orientation of the camera 15 with respect to the observation object S matches the amount-of-curvature information is created.

Furthermore, for example, when the library data called from the auxiliary storage apparatus 27 is roll angle information, based on a present roll angle of the scope axis and the roll angle information, the main controller 21 determines a position/attitude command around a pivot point for matching the roll angle of the scope axis with the roll angle information.

Next, the position/attitude controller 25 determines a drive amount of each joint of the robot arm 5 based on the position/attitude command. Due to each joint of the robot arm 5 moving according to each angle command, an endoscopic field of view in which an inclination of the observation object S around the optical axis of the camera 15 matches the roll angle information is created.

Next, a forceps operation by an assistant, an expansion operation, and the like are performed in the created endoscopic field of view (step S5).

Next, when a transition is made to an actual treatment performed by an operator, the main controller 21 recognizes the observation object S and specifies a procedural scene by processing image information newly acquired by the camera 15 (step S6). In addition, after the main controller 21 calls library data associated with the specified procedural scene from the auxiliary storage apparatus 27 (step S7), the main controller 21 creates an endoscopic field of view for treatment by controlling the electric scope 3 and the robot arm based on the called library data (step S8). Since a method of creating an internal use field of view of treatment is similar to the method of the endoscopic field of view for expansion, a description will be omitted.

Once the endoscopic field of view for treatment is created, tracking of a treatment tool by the operator is started in the created endoscopic field of view (step S9).

As described above, with the endoscope system 1, the procedure support method, and the procedure support program according to the present embodiment, after library data associated with any procedural scene is called from the auxiliary storage apparatus 27 by an actuation of the control apparatus 7, at least one of the robot arm 5 or the curved part 17 of the electric scope 3 is controlled based on the library data. In this case, since each piece of library data includes relative parameters related to relative positions and attitudes of the electric scope 3 with respect to the camera 15 and the observation object S, an operator can be provided with an endoscopic field of view which accommodates both a procedural scene and the observation object S. Therefore, the operator's effort can be reduced and treatment can be performed smoothly.

In the present embodiment, a case where any one of distance information, amount-of-curvature information, and roll angle information is called as library data from the auxiliary storage apparatus 27 has been described as an example. Alternatively, a plurality of pieces of library data may be called at the same time.

For example, when all three of the distance information, the amount-of-curvature information, and the roll angle information are called as library data from the auxiliary storage apparatus 27, based on a joint control command and a position/attitude command, the position/attitude controller 25 determines a drive amount of each joint required to respectively match the distance between the camera 15 and the observation object S, the amount of curvature of the curved part 17, and the roll angle of the scope axis with the distance information, the amount-of-curvature information, and the roll angle information.

In addition, based on an angle of curvature command, the field of view controller 23 determines an angle of curvature of the curved part 17 required to respectively match the distance between the camera 15 and the observation object S and the angle of curvature of the curved part 17 with the distance information and the amount-of-curvature information.

Next, due to each joint of the robot arm 5 moving according to each angle command and the curved part 17 of the electric scope 3 curving according to the motor angle command, an endoscopic field of view is created in which the distance between the camera 15 and the observation object S, the orientation of the camera 15 with respect to the observation object S, and the inclination of the observation object S around the optical axis of the camera 15 respectively match the distance information, the amount-of-curvature information, and the roll angle information.

Once all three of the distance between the camera 15 and the observation object S, the amount of curvature of the electric scope 3, and the roll angle of the scope axis are determined, the position and the attitude of the electric scope 3 are uniquely determined based on the observation object S. Therefore, there is no need to register data for each patient in advance and an endoscopic field of view can be created using same physical quantities even with respect to different patients.

The present embodiment can be modified as follows.

While the main controller 21 recognizes the observation object S by processing an endoscopic image in the present embodiment, alternatively, for example, the operator may designate an observation object using a user interface (UI) such as the headset 35, the hand switch 37 or the foot switch 39. In addition, instead of the main controller 21 specifying a procedural scene, the operator may specify a procedural scene.

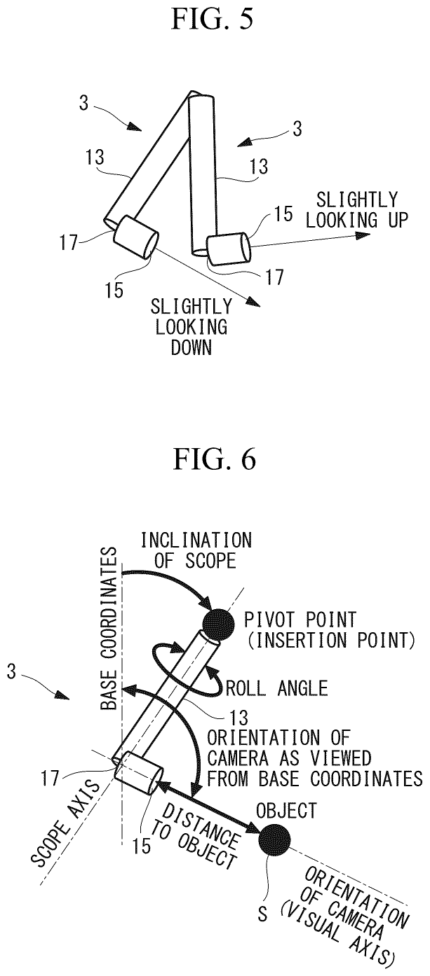

Furthermore, in the present embodiment, an amount of curvature of the curved part 17 of the electric scope 3 has been exemplified and described as library data. In this case, for example, as shown in FIG. 5, even when the amount of curvature of the curved part 17 is the same, a degree by which the camera 15 of the electric scope 3 looks up to or looks down to the observation object S changes depending on an inclination of the electric scope 3.

In consideration thereof, for example, as shown in FIG. 6, information indicating an orientation of the camera 15 as viewed from base coordinates with a center of a base of the robot arm 5 as an origin or, in other words, an orientation of the visual axis based on the base coordinates may be adopted as the library data in place of the amount-of-curvature information. Hereinafter, this information will be referred to as base coordinate-view orientation information.

The orientation of the camera 15 as viewed from the base coordinates is determined by a sum of an inclination of the electric scope 3 with respect to the base coordinates and an amount of curvature of the curved part 17. By matching the orientation of the camera 15 as viewed from the base coordinates in a given procedural scene with the base coordinate-view orientation information associated with the procedural scene, even if the inclination of the electric scope 3 changes according to a change in a position of biological tissue due to individual variability or the like, the orientation of the camera 15 can be prevented from changing. Therefore, an endoscopic field of view at the time of setting and registration can be more readily and more accurately reproduced.

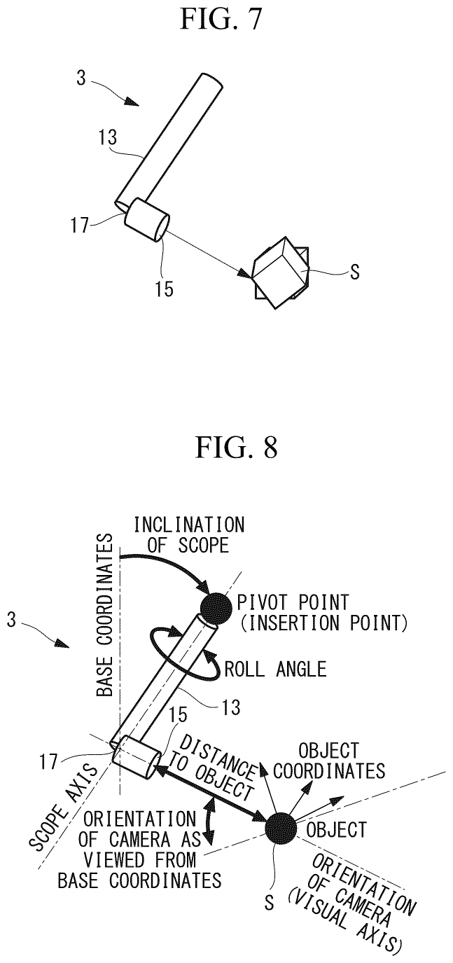

In addition, in a case where amount-of-curvature information of the curved part 17 and orientation information of the visual axis are adopted as library data, for example, as shown in FIG. 7, when an attitude of the observation object S changes due to an operation of forceps by an assistant or the like, an angle at which the camera 15 looks into the observation object S may relatively change.

In consideration thereof, for example, as shown in FIG. 8, information indicating an orientation of the camera 15 with respect to the observation object S or, in other words, an orientation of the visual axis as viewed from a coordinate of the observation object S may be adopted as the library data. Hereinafter, this information will be referred to as object-view orientation information. By matching the orientation of the camera 15 as viewed from the observation object S in a given procedural scene with object-view orientation information associated with the procedural scene, the orientation of the camera 15 is determined in accordance with an attitude of an organ or biological tissue. Therefore, even if an attitude or an orientation of the biological tissue changes due to an operation of forceps by an assistant or the like, a same endoscopic field of view or, in other words, a same angle at which the biological tissue is looked into as during setting and registration can be created.

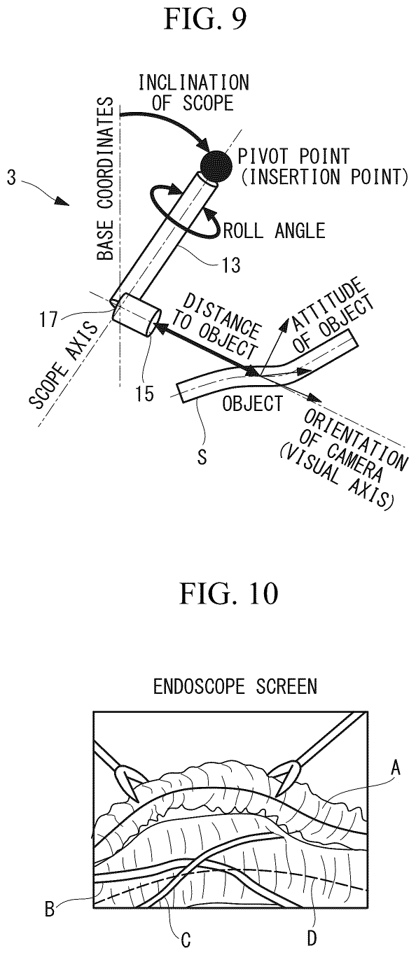

In addition, when scope-axis roll angle information is adopted as library data, for example, as shown in FIG. 9, an inclination around the optical axis of the camera 15 in a direction in which an elongated observation object S such as an intestine or a blood vessel runs may be recognized. Furthermore, a parameter which orients the direction in which the intestine or the blood vessel runs in a horizontal direction or a vertical direction of a screen of the monitor 11 may be set as roll angle information.

In an actual procedure, for example, as shown in FIG. 10, a field of view is adjusted in order to orient the direction in which the intestine or the blood vessel runs or, in other words, a direction in which a dissection line extends in the horizontal direction or the vertical direction of the screen of the monitor 11. Due to control based on library data, by orienting the direction in which the intestine or the blood vessel runs in the horizontal direction or the vertical direction of the screen of the monitor 11, an endoscopic field of view which better accommodates individual variability among patients can be obtained. In FIG. 10, reference sign A denotes the intestine, reference sign B denotes a nerve fascicle, reference sign C denotes an IMA (Inferior Mesenteric Artery), and reference sign D denotes a dissection line.

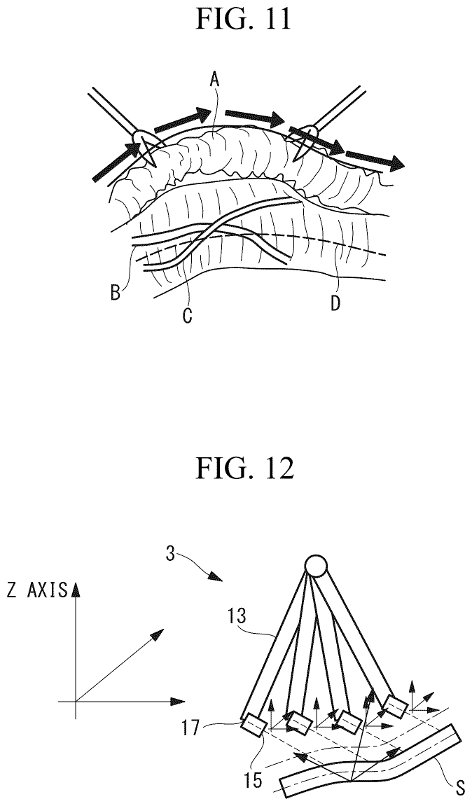

In this case, for example, as shown in FIG. 11, after approximating the direction in which the elongated observation object S such as an intestine or a blood vessel runs with a curve, gradients at a plurality of points are calculated and an average angle of the gradients is calculated. In addition, the direction in which the elongated observation object S runs may be matched with the average angle by respectively adjusting the roll angle of the electric scope 3 and the angle of curvatures of the curved part 17 in the UD (up down) direction and the LR (left right) direction.

Furthermore, by inputting or detecting a coordinate of the patient or the operating table in advance, for example, as shown in FIG. 12, the roll angle of the electric scope 3 may be matched with the coordinate of the patient or the operating table in a Z-axis direction (usually, vertically upward).

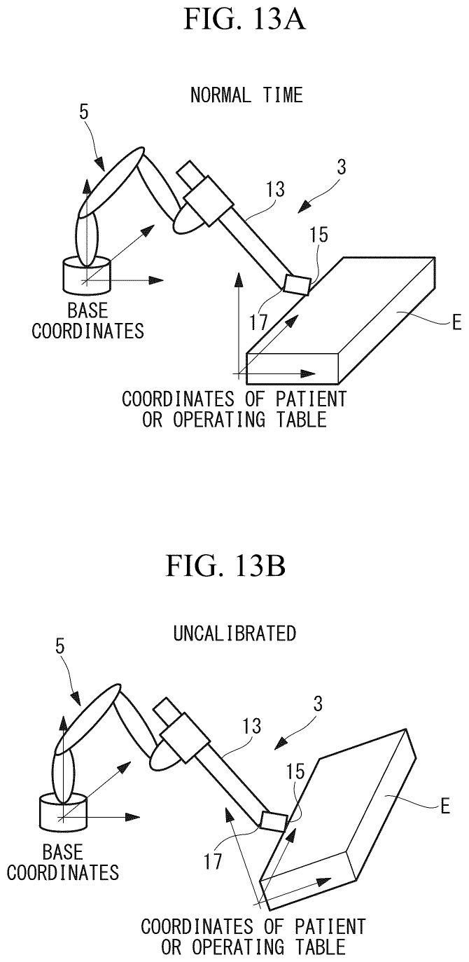

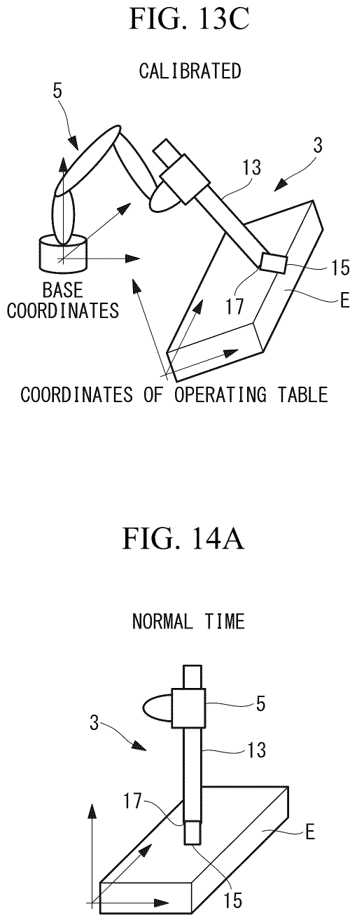

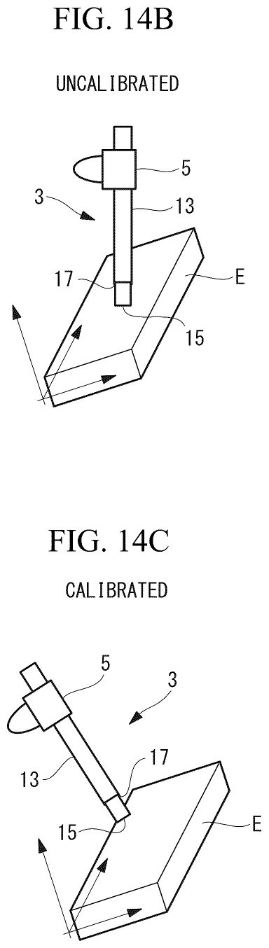

In addition, in the present embodiment, library data or the base coordinates may be calibrated in accordance with an inclination of the patient himself/herself or an inclination of the operating table on which the patient lies. For example, as shown in FIGS. 13A to 13C, after detecting an inclination of the patient or the operating table, the base coordinates may be corrected by adding the detected inclination to the base coordinates. Furthermore, for example, as shown in FIGS. 14A to 14C, after detecting an inclination of the patient or the operating table, library data may be corrected by adding the detected inclination to the library data. In FIGS. 13A to 13C and FIGS. 14A to 14C, reference sign E denotes an operating table or a patient.

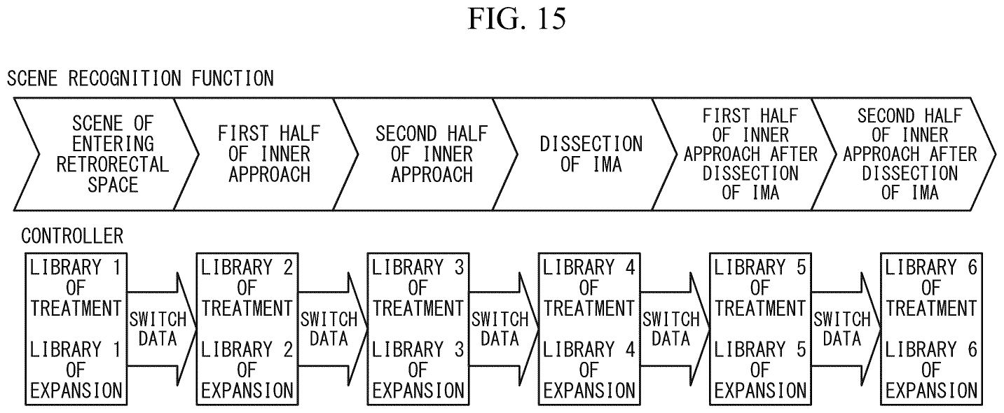

In addition, in the present embodiment, a flow of procedures may be programmed in advance and switching among pieces of library data associated with each procedural scene may be performed in accordance with a treatment step or, in other words, a procedural scene. In this case, for example, the main controller 21 may switch among pieces of library data by having an AI (Artificial Intelligence) judge a treatment step as shown in FIG. 15.

A treatment step is, for example, an anatomical location being treated or an operation by an operator such as an incision. An AI may estimate a treatment step based on the anatomical location and an operation by the operator such as an incision. Conceivable examples of the operation by the operator include a dissection of a specific blood vessel, a hemostatic operation with respect to a hemorrhage, clipping of a vascular channel, a compression operation of an organ, and fluorescent observation.

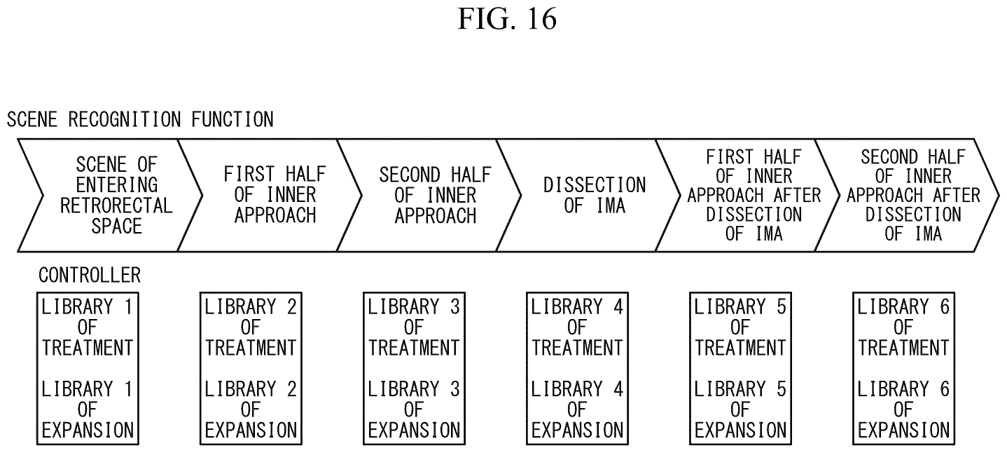

In addition, a treatment step may be recognized by the operator. For example, a treatment step may be specified by the operator by using a UI (User Interface) such as voice or a button operation. In this case, for example, as shown in FIG. 16, the main controller 21 may switch among pieces of library data based on the operator specifying a treatment step by UI.

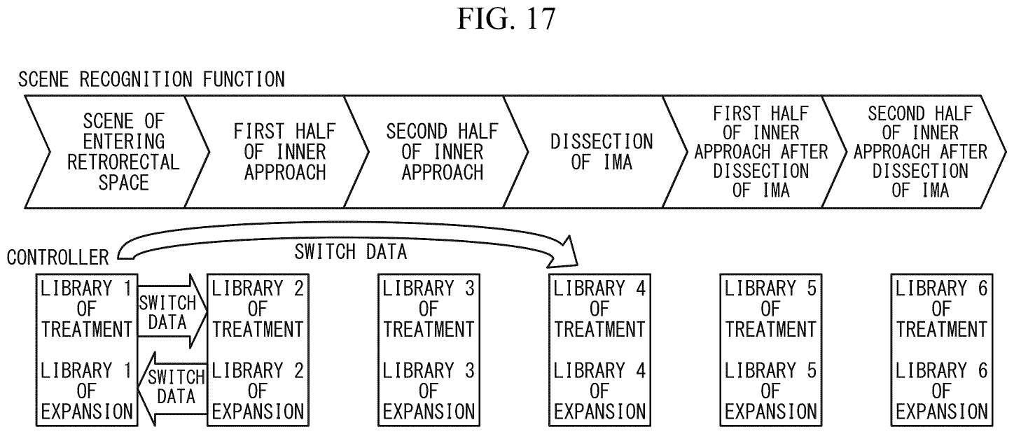

Furthermore, for example, as shown in FIG. 17, the main controller 21 may switch to library data associated with a designated procedural scene based on an instruction by the operator such as “Proceed to next treatment step”, “Return to previous treatment step”, or “Proceed to step of dissection of the IMA”.

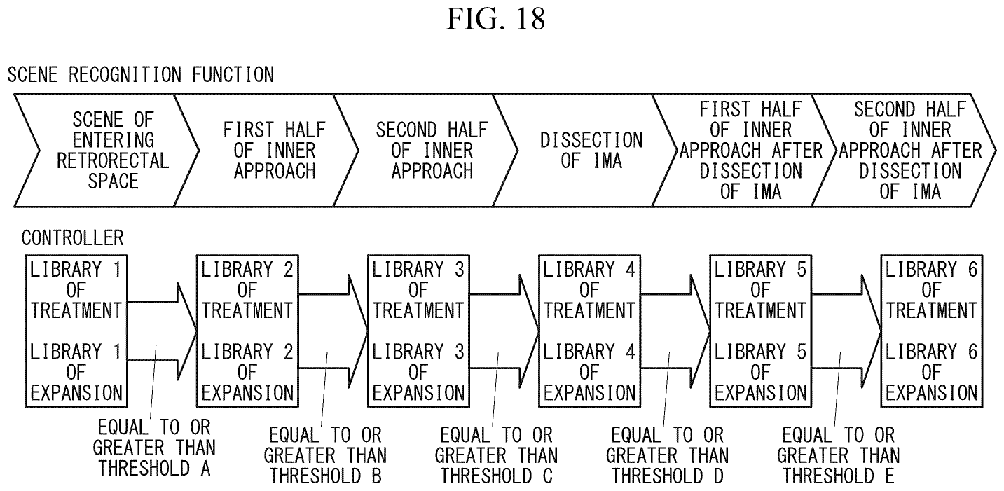

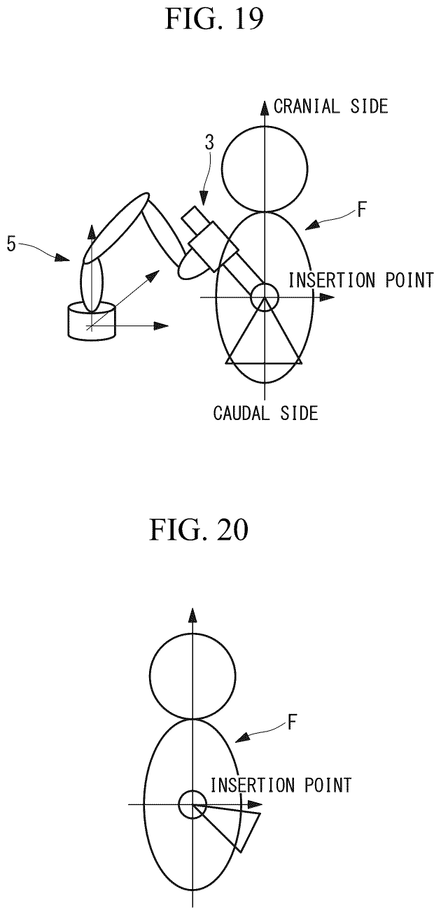

In addition, since the position of the camera 15 with respect to the patient changes as the treatment progresses, for example, switching among pieces of library data may be performed based on the position and the attitude of the camera 15 with respect to the patient. As shown in FIGS. 18 and 19, for example, in a case where an insertion point is defined as an origin, a cranial side of a patient is defined as 0 degrees, and a caudal side of the patient is defined as 180 degrees, the main controller 21 may set library data associated with a “scene of entering retrorectal space” when the orientation of the camera 15 is under a threshold A indicating a treatment within a triangular frame in FIG. 19 (for example, the orientation of the camera 15 is from 210 to 150 degrees). In FIG. 19, reference sign F denotes a patient. A similar description applies to FIG. 20.

In addition, as shown in FIGS. 18 and 20, the main controller 21 may switch to library data associated with a “first half of inner approach” when the orientation of the camera 15 is equal to or greater than the threshold A and under a threshold B indicating a treatment within a triangular frame in FIG. 20 (for example, the orientation of the camera 15 is from 150 to 120 degrees).

The main controller 21 may adopt biological tissue recognized by tissue recognition using an AI as the observation object S. By learning tissue such as a blood vessel or an organ by machine learning or the like in advance, when a learned tissue appears in an image, the main controller 21 can automatically set the tissue as the observation object S.

In addition, the main controller 21 may recognize biological tissue designated by an operator using a UI as the observation object S. In this case, the operator may use a UI such as a touch panel to designate a sanctions organization to be adopted as the observation object S or a point on biological tissue indicated by a leading end of forceps or the like may be stored and the stored biological tissue may be adopted as the observation object S. Accordingly, since learning need not be performed in advance, various biological tissues can be readily set as the observation object S.

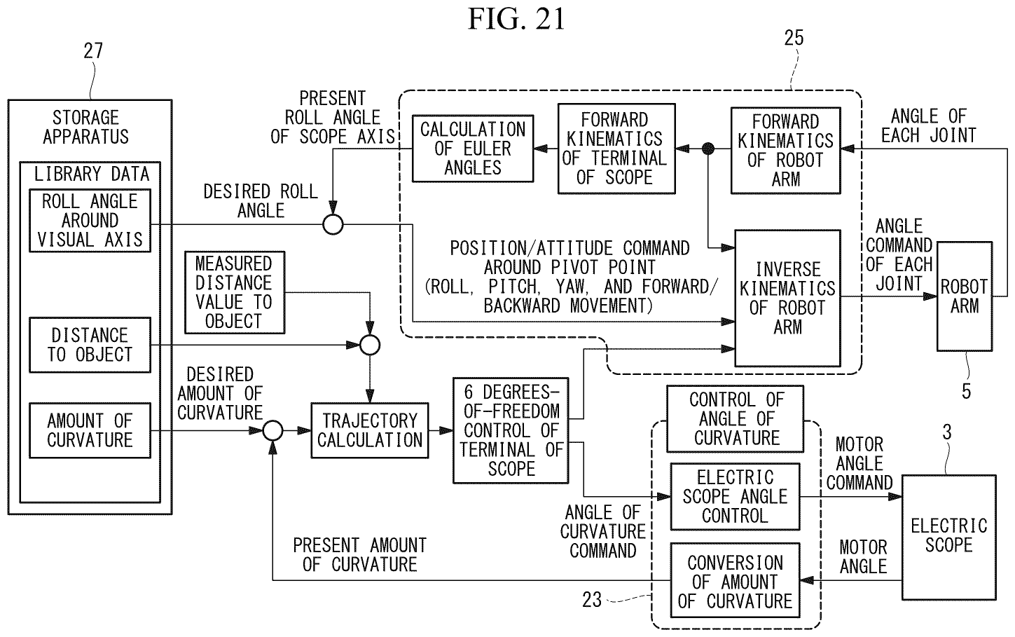

In addition, while the scope-axis roll angle has been exemplified and described as library data in the present embodiment, for example, a roll angle around the optical axis of the camera 15 on a leading end side of the curved part 17 or, in other words, a roll angle around the visual axis may be adopted instead of the scope-axis roll angle. In this case, for example, as shown in FIG. 21, the position/attitude controller 25 may calculate Euler angles (roll, pitch, and yaw) based on the angle of each joint of the robot arm 5 using forward kinematics of a terminal of the electric scope 3 in addition to forward kinematics of the robot arm 5.

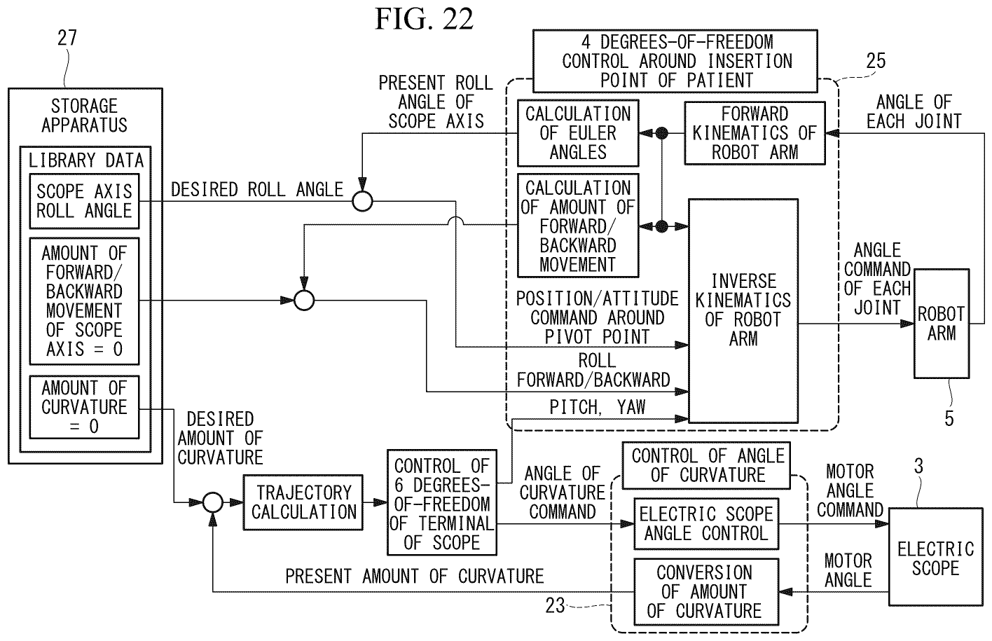

Furthermore, the present embodiment can also be applied when performing an expansion operation in a state where a downward endoscopic field of view is created by retracting the electric scope 3 to a vicinity of an insertion point to the patient such as a vicinity of a trocar. In this case, for example, as shown in FIG. 22, amount-of-curvature information indicating that the amount of curvature of the curved part 17 is zero, scope-axis forward/backward movement amount information indicating that the amount of forward/backward movement of the electric scope 3 is zero, and scope-axis roll angle information of which the roll angle takes a desired value may be adopted as library data. A state where the amount of curvature of the curved part 17 is zero is a state where the curved part 17 is oriented in the longitudinal axis direction of the insertion portion 13. A state where the amount of forward/backward movement of the electric scope 3 is zero is a state where the electric scope 3 is positioned in the vicinity of the insertion point to the patient.

Due to such control based on library data, after the amount of curvature of the curved part 17 is restored to zero, the electric scope 3 is retracted until the amount of forward/backward movement becomes zero or, in other words, retracted to an extraction limit and the roll angle of the electric scope 3 is adjusted. By retracting the electric scope 3 to the vicinity of the trocar in a state where the curved part 17 extends in a straight line along the longitudinal axis of the insertion portion 13, a completely downward endoscopic field of view can be created. In this case, the position/attitude controller 25 may calculate a present amount of forward/backward movement of the electric scope 3 based on the angle of each joint of the robot arm 5 using forward kinematics of the robot arm 5. The calculated present amount of forward/backward movement of the electric scope 3 is stored in the main controller 21.

### Second Embodiment

Next, an endoscope system, a procedure support method, and a procedure support program according to a second embodiment of the present disclosure will be described.

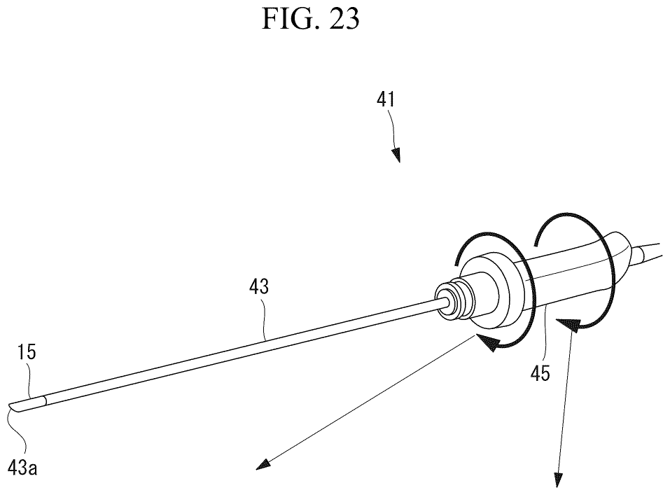

The endoscope system 1 according to the present embodiment differs from the first embodiment in that, as shown in FIG. 23, the endoscope system 1 includes an oblique-viewing endoscope (endoscope) 41 not equipped with the curved part 17 instead of the electric scope 3.

Hereinafter, portions which share common configurations with the endoscope system 1, the procedure support method, and the procedure support program according to the first embodiment will be assigned the same reference signs and descriptions thereof will be omitted.

The oblique-viewing endoscope 41 includes an elongated lens barrel unit 43 to be inserted into a body cavity of the patient and the camera (imaging optical system) 15 provided at a tip portion of the lens barrel unit 43. The oblique-viewing endoscope 41 is arranged in a state where the optical axis of the camera 15 is inclined with respect to a longitudinal axis (central axis) of the lens barrel unit 43. The lens barrel unit 43 includes a tip surface 43a which is inclined with respect to the longitudinal axis of the lens barrel unit 43 and which is orthogonal to the optical axis of the camera 15. Reference sign 45 denotes a mounting portion to be supported by the robot arm 5. In addition, the oblique-viewing endoscope 41 includes a ranging function.

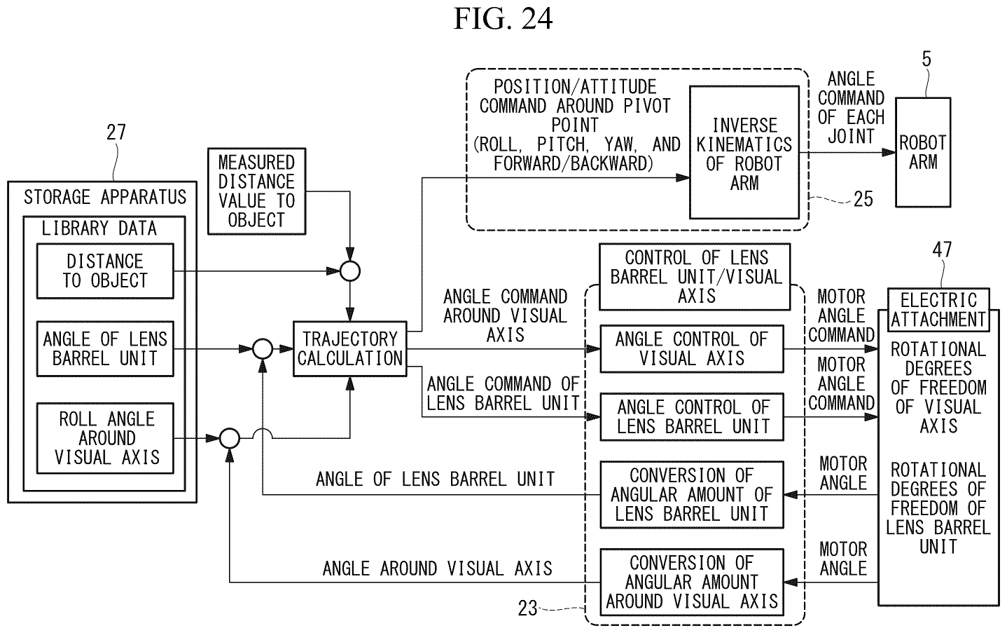

As shown in FIG. 24, the oblique-viewing endoscope 41 has a built-in electric attachment (field-of-view direction changing unit) 47. The electric attachment 47 is constituted of a lens barrel unit motor which rotates the lens barrel unit 43 around the longitudinal axis, a visual axis motor (both motors not illustrated) which rotates and drives an image rotator (not illustrated) that rotates the visual axis of the camera 15 around an axial line, and the like.

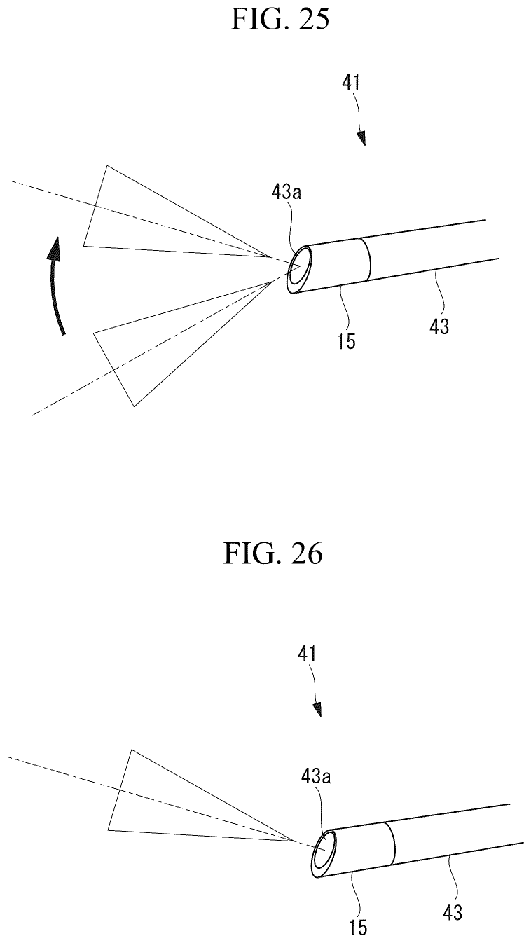

Due to the lens barrel unit 43 rotating around the longitudinal axis by being driven by the lens barrel unit motor, as shown in FIG. 25, a direction in which the tip surface 43a at a certain angle with respect to the longitudinal axis of the lens barrel unit 43 is oriented and a direction of the optical axis of the camera 15 or, in other words, a field-of-view direction of the oblique-viewing endoscope 41 can be changed around the longitudinal axis of the lens barrel unit 43.

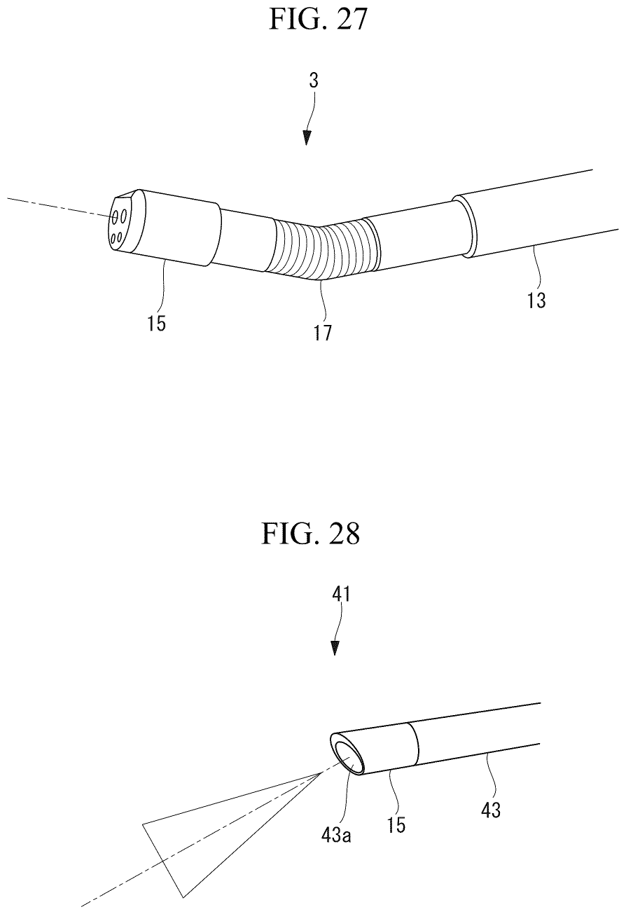

Rotating the lens barrel unit 43 of the oblique-viewing endoscope 41 around the longitudinal axis is equivalent to changing a distribution of upward, downward, leftward, and rightward in a direction of curvature of the curved part 17 while keeping the amount of curvature constant in the electric scope 3. For example, as shown in FIG. 26, a look-up field of view when the tip surface 43a of the oblique-viewing endoscope 41 and the camera 15 are made to face obliquely upward is equivalent to the field of view when the curved part 17 of the electric scope 3 is curved by around 30 degrees in an upward direction as shown in FIG. 27. In addition, for example, as shown in FIG. 28, a look-down field of view when the tip surface 43a of the oblique-viewing endoscope 41 and the camera 15 are made to face obliquely downward is equivalent to the field of view when the curved part 17 of the electric scope 3 is curved by around 30 degrees in a downward direction as shown in FIG. 29.

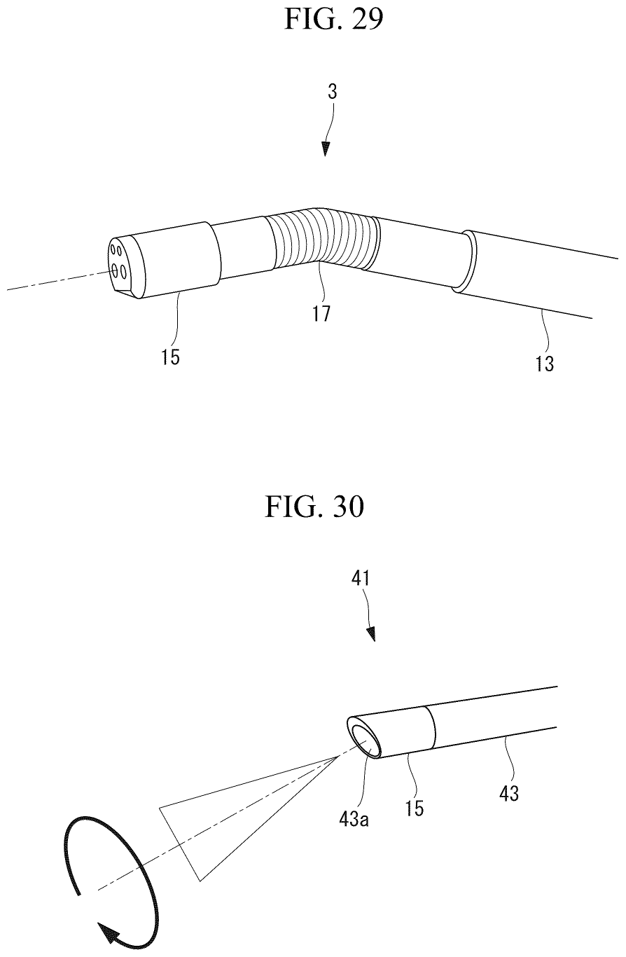

In addition, for example, by rotating the visual axis of the camera 15 around the axial line due to being driven by the visual axis motor as shown in FIG. 30, a vertical direction of an endoscopic image which is acquired by the camera 15 can be rotated around the visual axis.

The plurality of pieces of library data stored in the auxiliary storage apparatus 27 include at least one relative parameter related to a relative position and a relative attitude of the oblique-viewing endoscope 41 with respect to the camera 15 and the observation object S to be photographed by the camera 15. Examples of the relative parameter include distance information of a distance between the camera 15 and the observation object S, rotational angle information around the longitudinal axis of the lens barrel unit 43, and roll angle information around the visual axis. The roll angle information around the visual axis represents an angle around the axial line of the visual axis of the camera 15. Hereinafter, these pieces of information will be referred to as distance information, lens-barrel-unit angle information, and roll angle-around-visual axis information. In the auxiliary storage apparatus 27, at least any one of distance information, lens-barrel-unit angle information, and roll angle-around-visual axis information for realizing each endoscopic field of view set in advance for each procedural scene is stored in association with each procedural scene.

The procedure support method includes a step of changing at least any one of an angle around the longitudinal axis of the lens barrel unit 43 and a position and an attitude of the oblique-viewing endoscope 41 based on the called library data instead of the step of changing at least any one of the angle of the camera 15 and the position and the attitude of the electric scope 3 (steps S4 and S8 in the first embodiment). The procedure support program causes the steps to be executed by the respective controllers 21, 23, and 25 of the control apparatus 7.

For example, when the control apparatus 7 calls the distance information from the auxiliary storage apparatus 27, the control apparatus 7 matches an actual distance between the camera 15 and the observation object S which is calculated from image information of the observation object S with the called distance information by controlling at least one of the electric attachment 47 or the robot arm 5. Specifically, as shown in FIG. 24, the main controller 21 calculates a trajectory for matching the distance between the camera 15 and the observation object S with the distance information by comparing a present measured distance value to the observation object S and the called distance information. In addition, after distribution to a position/attitude command indicating an amount of change of a position and an attitude around the pivot point, an angle command around the visual axis, and an angle command of the lens barrel unit, the main controller 21 inputs the position/attitude command to the position/attitude controller 25 and inputs the angle command around the visual axis and the angle command of the lens barrel unit to the field of view controller 23.

According to the position/attitude command, the position/attitude controller 25 uses inverse kinematics of the robot arm 5 to determine a drive amount of each joint required to match the distance between the camera 15 and the observation object S with the distance information. The determined drive amount of each joint is inputted to each motor of the robot arm 5 as an angle command for each joint (endoscope operation command).

On the other hand, according to the angle command around the visual axis and the angle command of the lens barrel unit, the field of view controller 23 determines a rotational angle around the visual axis and a rotational angle of the lens barrel unit 43 required to match the distance between the camera 15 and the observation object S with the distance information. The determined rotational angle around the visual axis and the determined rotational angle of the lens barrel unit 43 are respectively inputted to the visual axis motor and the lens barrel unit motor of the electric attachment 47 as motor angle commands.

Due to each joint of the robot arm 5 moving according to each angle command and each motor of the electric attachment 47 providing drive according to each motor angle command, an endoscopic field of view in which the distance between the camera 15 and the observation object S matches the distance information is obtained.

In addition, when lens-barrel-unit angle information is called from the auxiliary storage apparatus 27, the control apparatus 7 first calculates a trajectory for matching the rotational angle of the lens barrel unit 43 with the lens-barrel-unit angle information by having the main controller 21 compare a present angle of the lens barrel unit 43 around the longitudinal axis and the called lens-barrel-unit angle information. Furthermore, the position/attitude controller 25 and the field of view controller 23 control at least one of the electric attachment 47 or the robot arm 5. Accordingly, the angle of the lens barrel unit 43 around the longitudinal axis as calculated from the image information of the observation object S is matched with the called lens-barrel-unit angle information. Since control by the position/attitude controller 25 and the field of view controller 23 is similar to the case of distance information, a description thereof will be omitted.

In addition, when roll angle information around the visual axis is called from the auxiliary storage apparatus 27, the control apparatus 7 first calculates a trajectory for matching the angle around the axial line of the visual axis with the roll angle information around the visual axis by having the main controller 21 compare a present angle around the axial line of the visual axis of the camera 15 and the called roll angle information around the visual axis. Furthermore, by having the position/attitude controller 25 and the field of view controller 23 control at least one of the electric attachment 47 or the robot arm 5, the angle around the axial line of the visual axis of the camera 15 which is calculated from image information of the observation object S is matched with the called roll angle information around the visual axis. Since control by the position/attitude controller 25 and the field of view controller 23 is similar to the case of distance information, a description thereof will be omitted.

When the field of view controller 23 drives the lens barrel unit motor of the electric attachment 47, the field of view controller 23 calculates a present angular amount of the lens barrel unit 43 by converting a motor angle of the lens barrel unit motor into an angular amount around the longitudinal axis of the lens barrel unit 43. In addition, when the field of view controller 23 drives the visual axis motor of the electric attachment 47, the field of view controller 23 calculates a present angular amount around the axial line of the visual axis by converting a motor angle of the visual axis motor into an angular amount around the axial line of the visual axis. The calculated present angular amount of the lens barrel unit 43 and the calculated present angular amount around the axial line of the visual axis are respectively stored in the main controller 21.

With the endoscope system 1, the procedure support method, and the procedure support program according to the present embodiment, since each piece of library data includes relative parameters related to relative positions and attitudes of the camera 15 of the oblique-viewing endoscope 41 and the observation object S, an operator can be provided with an endoscopic field of view which accommodates both a procedural scene and the observation object S. In this case, for example, when the angle of the lens barrel unit 43 around the longitudinal axis is changed based on the lens-barrel-unit angle information, the field-of-view direction of the oblique-viewing endoscope 41 is switched to obliquely upward, obliquely downward, or the like due to the field-of-view direction of the oblique-viewing endoscope 41 with a certain angle with respect to the longitudinal axis of the lens barrel unit 43 changing around the longitudinal axis of the lens barrel unit 43. Accordingly, the field-of-view direction of the oblique-viewing endoscope 41 can be oriented toward a desired observation object by simply rotating the lens barrel unit 43 around the longitudinal axis. As a result, even when the oblique-viewing endoscope 41 is adopted as the endoscope, an observation object designated by an operator can be readily placed inside the endoscopic field of view.

### Third Embodiment

Next, an endoscope system, a procedure support method, and a procedure support program according to a third embodiment of the present disclosure will be described.

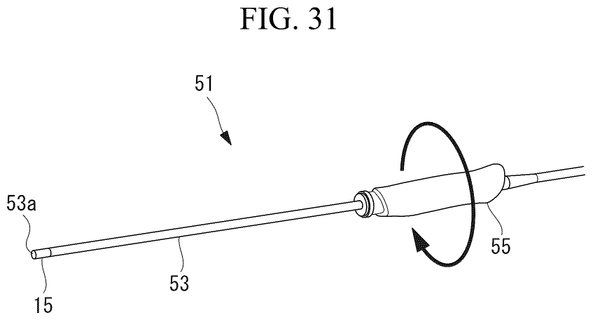

The endoscope system 1 according to the present embodiment differs from the first and second embodiments in that, as shown in FIG. 31, the endoscope system 1 includes a forward-viewing endoscope (endoscope) 51 not equipped with the curved part 17 instead of the electric scope 3 and the oblique-viewing endoscope 41.

Hereinafter, portions which share common configurations with the endoscope system 1, the procedure support method, and the procedure support program according to the first and second embodiments will be assigned the same reference signs and descriptions thereof will be omitted.

The forward-viewing endoscope 51 includes an elongated lens barrel unit 53 to be inserted into a body cavity of the patient and the camera (imaging optical system) 15 provided at a tip portion of the lens barrel unit 53. The forward-viewing endoscope 51 is arranged in a state where a longitudinal axis (central axis) of the lens barrel unit 53 matches with the optical axis of the camera 15. The lens barrel unit 53 includes a tip surface 53a which is orthogonal to the longitudinal axis of the lens barrel unit 53 and the optical axis of the camera 15. Reference sign 55 denotes a mounting portion to be supported by the robot arm 5. In addition, the forward-viewing endoscope 51 includes a ranging function.

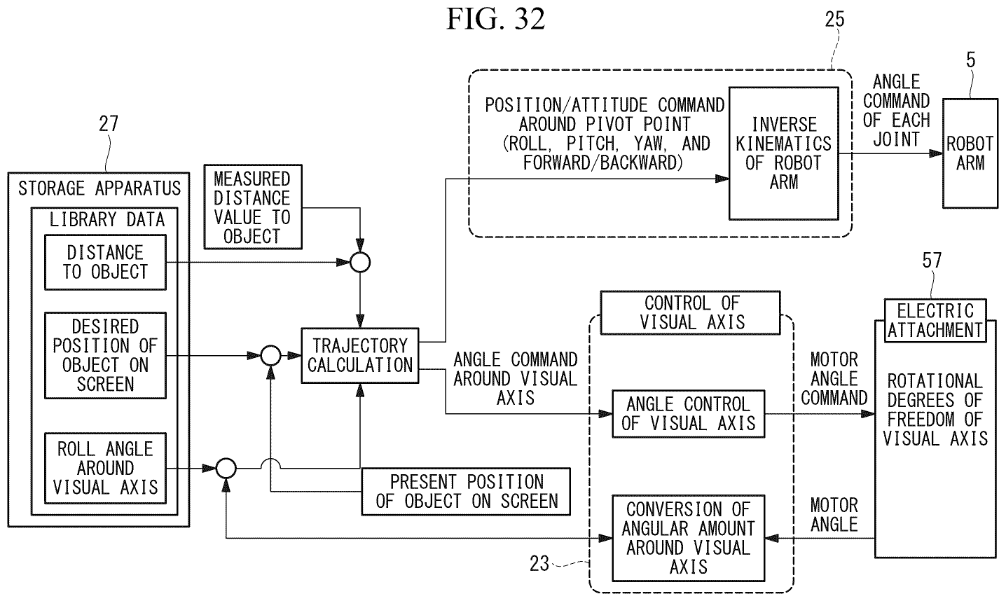

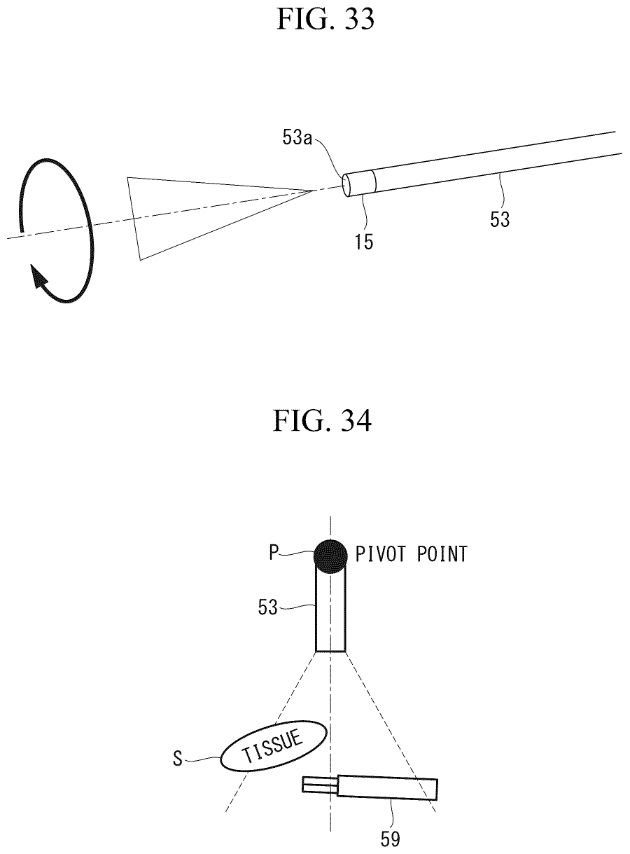

As shown in FIG. 32, the forward-viewing endoscope 51 has a built-in electric attachment 57. The electric attachment 57 is constituted of a visual axis motor which rotates the visual axis of the camera 15 around an axial line or, in other words, around the longitudinal axis of the lens barrel unit 53, and the like. By rotating the visual axis of the camera 15 around the axial line as shown in FIG. 33, the forward-viewing endoscope 51 can rotate a vertical direction of an endoscopic image which is acquired by the camera 15 around the visual axis.

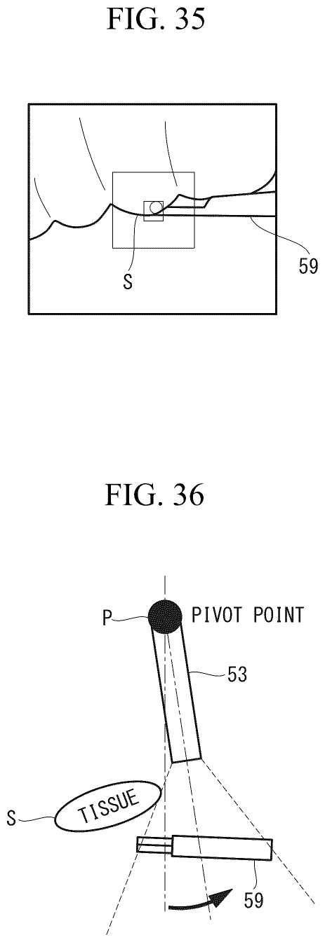

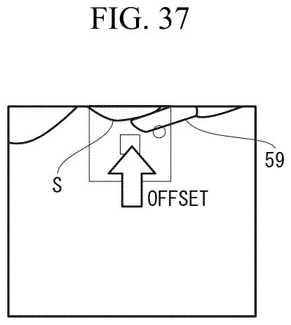

The robot arm 5 functions as a field-of-view direction changing unit which changes an angle around a pivot axis (rotational axis) which is orthogonal to the longitudinal axis of the lens barrel unit 53 in accordance with a change in a position where the observation object S is captured on the screen of the monitor 11. For example, when the position where the observation object in biological tissue or the like is captured is changed to an end in an upper part of the screen of the monitor 11 from a state where the observation object is captured at center of the screen, as shown in FIG. 34 and FIG. 36, an angle around a pivot point P which is the mounting portion 55 of the forward-viewing endoscope 51 is changed. Accordingly, as a result of the field-of-view direction of the forward-viewing endoscope 51 being changed to an orientation in which the observation object is captured at an end of the upper part of the screen, the position of the observation object on the screen of the monitor 11 is offset in a direction which intersects with the optical axis of the camera 15 as shown in FIG. 35 and FIG. 37.

When the observation object is captured at the center of the endoscopic field of view as shown in FIG. 34 and FIG. 35, there is a location which is hidden and hard to view although the observation object is positioned at the center of the screen. In contrast, when the observation object is captured at the end of an angle of view as shown in FIG. 36 and FIG. 37, the observation object is positioned at the end of the screen and the location previously hidden and hard to view becomes visible. In other words, by changing the field-of-view direction of the forward-viewing endoscope 51 to a direction in which the observation object is captured at the end of the angle of view, an endoscopic field of view of an angle of looking into the observation object can be created. In FIG. 34 and FIG. 36, reference sign 59 denotes a treatment tool and reference sign S denotes the observation object.

The plurality of pieces of library data stored in the auxiliary storage apparatus 27 include at least one relative parameter related to a relative position and a relative attitude of the forward-viewing endoscope 51 with respect to the camera 15 and the observation object S to be photographed by the camera 15. Examples of the relative parameter include distance information of a distance between the camera 15 and the observation object S, position information on a position where the observation object is captured on the screen, and roll angle information around the visual axis. Hereinafter, these pieces of information will be referred to as distance information, object position information, and roll angle-around-visual axis information. In the auxiliary storage apparatus 27, at least any one of distance information, object position information, and roll angle-around-visual axis information for realizing each endoscopic field of view set in advance for each procedural scene is stored in association with each procedural scene. For example, a position where the observation object is desirably captured on the screen of the monitor 11 may be set as object position information by an audio operation using a headset 35, a button operation using a hand switch 37, or the like.

The procedure support method includes a step of changing at least any one of an angle around the pivot point P of the forward-viewing endoscope 51 and a position and an attitude of the forward-viewing endoscope 51 based on the called library data instead of the step of changing at least any one of the angle of the camera 15 and the position and the attitude of the electric scope 3 (steps S4 and S8 in the first embodiment) and the procedure support program causes the step to be executed by the respective controllers 21, 23, and 25 of the control apparatus 7.

For example, when the control apparatus 7 calls the distance information from the auxiliary storage apparatus 27, the control apparatus 7 matches an actual distance between the camera 15 and the observation object S which is calculated from image information of the observation object S with the called distance information by controlling at least one of the electric attachment 57 or the robot arm 5. Specifically, as shown in FIG. 32, the main controller 21 calculates a trajectory for matching the distance between the camera 15 and the observation object S with the distance information by comparing a present measured distance value to the observation object S and the called distance information. In addition, after distribution to a position/attitude command indicating an amount of change of a position and an attitude around the pivot point and an angle command around the visual axis, the main controller 21 inputs the position/attitude command to the position/attitude controller 25 and inputs the angle command around the visual axis to the field of view controller 23.

According to the position/attitude command, the position/attitude controller 25 uses inverse kinematics of the robot arm 5 to determine a drive amount of each joint required to match the distance between the camera 15 and the observation object S with the distance information. The determined drive amount of each joint is inputted to each motor of the robot arm 5 as an angle command for each joint (endoscope operation command).

On the other hand, according to the angle command around the visual axis, the field of view controller 23 determines a rotational angle around the visual axis required to match the distance between the camera 15 and the observation object S with the distance information. The determined rotational angle around the visual axis is inputted to the visual axis motor of the electric attachment 57 as a motor angle command.

Due to each joint of the robot arm 5 moving according to each angle command and the visual axis motor of the electric attachment 57 rotating according to the motor angle command, an endoscopic field of view in which the distance between the camera 15 and the observation object S matches the distance information is obtained.

In addition, when object position information is called from the auxiliary storage apparatus 27, the control apparatus 7 first calculates a trajectory for matching a position of the observation object on a screen of the monitor 11 with the object position information by having the main controller 21 compare a present position of the observation object on the screen of the monitor 11 with the called object position information. Furthermore, the position/attitude controller 25 and the field of view controller 23 control at least one of the electric attachment 57 or the robot arm 5. Accordingly, the position of the observation object on the screen as calculated from the image information of the observation object S is matched with the called object position information. Since control by the position/attitude controller 25 and the field of view controller 23 is similar to the case of distance information, a description thereof will be omitted.

In addition, when roll angle information around the visual axis is called from the auxiliary storage apparatus 27, the control apparatus 7 first calculates a trajectory for matching the angle around the axial line of the visual axis with the roll angle information around the visual axis by having the main controller 21 compare a present angle around the axial line of the visual axis of the camera 15 and the called roll angle information around the visual axis. Furthermore, by having the position/attitude controller 25 and the field of view controller 23 control at least one of the electric attachment 57 or the robot arm 5, the angle around the axial line of the visual axis of the camera 15 which is calculated from image information of the observation object S is matched with the called roll angle information around the visual axis. Since control by the position/attitude controller 25 and the field of view controller 23 is similar to the case of distance information, a description thereof will be omitted.

When the field of view controller 23 drives the visual axis motor of the electric attachment 57, the field of view controller 23 calculates a present angular amount around the axial line of the visual axis by converting a motor angle of the visual axis motor into an angular amount around the axial line of the visual axis. The calculated present angular amount is stored in the main controller 21.

With the endoscope system 1, the procedure support method, and the procedure support program according to the present embodiment, since each piece of library data includes relative parameters related to relative positions and attitudes of the camera 15 of the forward-viewing endoscope 51 and the observation object S, an operator can be provided with an endoscopic field of view which accommodates both a procedural scene and the observation object S. In this case, for example, when the position at which the observation object is captured on the screen of the monitor 11 based on the object position information is changed to an end of the screen, a field-of-view direction of the forward-viewing endoscope 51 is changed to a direction in which the observation object is captured at the end of an angle of view due to a change in the angle of the forward-viewing endoscope 51 around the pivot point P. Accordingly, an endoscopic field of view of an angle of looking into the observation object can be created. As a result, even when the forward-viewing endoscope 51 is adopted as the endoscope, an observation object designated by an operator can be readily placed inside the endoscopic field of view.

While embodiments of the present disclosure have been described in detail with reference to the drawings, specific configurations are not limited to the embodiments and the present disclosure includes design changes and the like which are made without departing from the scope of the disclosure. For example, the present disclosure is not limited to the disclosure applied to each embodiment and each modification described above and may be applied to embodiments created by appropriately combining the above embodiments and the above modifications without being particularly limited thereto.

In addition, for example, for each piece of library data, the auxiliary storage apparatus 27 may store the piece of library data together with metadata enabling the piece of library data to be specified. In this case, the main controller 21 may acquire metadata inputted by the operator according to a procedural scene or acquire metadata corresponding to a procedural scene which is specified by processing image information. Furthermore, the main controller 21 may call library data corresponding to the acquired metadata from the auxiliary storage apparatus 27.

In addition, for example, while the robot arm 5 which is a 6-axis articulated robot has been exemplified and described as an electric arm in the present embodiment, the electric arm need not have six degrees of freedom and may be a robot arm with fewer degrees of freedom. For example, a three-joint robot arm which is constituted of a roll axis and two pitch axes and of which a leading end is operable at 3 degrees of freedom may be adopted. In this case, for example, the robot arm may include an electric attachment for rotating the electric scope 3. Alternatively, a robot arm with a 4 degree-of-freedom configuration and having a roll joint at a leading end thereof may be adopted.

The above-described embodiments also lead to the following aspects.

An aspect of the present disclosure is an endoscope system comprising: an endoscope that comprises an imaging optical system for photographing an observation object; an electric arm that changes a position and an attitude of the endoscope while supporting the endoscope; a field-of-view direction changing unit capable of changing a field-of-view direction of the endoscope; a storage apparatus that stores a plurality of pieces of library data for realizing an endoscopic field of view, each piece of library data being associated with each procedural scene; and at least one processor, wherein each piece of library data comprises at least one relative parameter related to relative positions or attitudes of the imaging optical system and the observation object, and after calling the library data associated with any procedural scene from the storage apparatus, the processor controls at least one of the field-of-view direction changing unit or the electric arm based on the called library data.

According to the present aspect, due to an actuation of the processor, library data for realizing an endoscopic field of view associated with any procedural scene is called from the storage apparatus. In addition, the field-of-view direction changing unit and the electric arm are controlled based on the called library data. In this case, since each piece of library data includes relative parameters related to relative positions and attitudes of the imaging optical system of the endoscope and the observation object, an operator can be provided with an endoscopic field of view that accommodates both a procedural scene and the observation object. Therefore, the operator's effort can be reduced and treatment can be performed smoothly.

In the endoscope system according to the aspect described above, after specifying the procedural scene by processing image information acquired by the imaging optical system, the processor may call the library data associated with the specified procedural scene from the storage apparatus.

According to this configuration, the field-of-view direction changing unit and the electric arm are controlled based on the procedural scene specified by image processing by the processor. Therefore, the operator need not specify a procedural scene from image information and the operator's effort can be further reduced.

In the endoscope system according to the aspect described above, the relative parameter may be distance information between the imaging optical system and the observation object, and the processor may match a distance between the imaging optical system and the observation object as calculated from image information acquired by the imaging optical system with the distance information by controlling at least one of the field-of-view direction changing unit or the electric arm.

According to this configuration, the imaging optical system of the endoscope is arranged at a distance set in advance with respect to the observation object in accordance with both a procedural scene and the observation object. Accordingly, the observation object can be shown with a desired sense of distance.

In the endoscope system according to the aspect described above, the relative parameter may be orientation information of the imaging optical system with respect to the observation object, and the processor may match the orientation of the imaging optical system with respect to the observation object as calculated from image information acquired by the imaging optical system with the orientation information by controlling at least one of the field-of-view direction changing unit or the electric arm.

According to this configuration, the imaging optical system of the endoscope is arranged in an orientation set in advance with respect to the observation object in accordance with both a procedural scene and the observation object. Since the attitude of the imaging optical system is determined in accordance with an attitude of an organ or other observed tissue, even if there is individual variability, an endoscopic field of view set in advance can be more readily and accurately reproduced. In addition, even if an attitude or an orientation of the observed tissue changes due to an operation of forceps by an assistant or the like, the endoscopic field of view set in advance can be readily obtained.

In the endoscope system according to the aspect described above, the relative parameter may be inclination information around an optical axis of the imaging optical system of the observation object with an elongated shape which extends in a direction orthogonal to the optical axis, and the processor may match an inclination of the observation object with an elongated shape around the optical axis of the imaging optical system as calculated from image information acquired by the imaging optical system with the inclination information by controlling at least one of the field-of-view direction changing unit or the electric arm.

According to this configuration, a direction in which the intestine, a blood vessel, or the like extends with respect to the endoscopic field of view can be arranged in an orientation set in advance in accordance with both a procedural scene and the observation object. Accordingly, an endoscopic field of view which better accommodates the individual variability of a patient can be readily obtained.

In the endoscope system according to the aspect described above, the field-of-view direction changing unit may be a curved part capable of changing an angle of the imaging optical system in the endoscope. In such a case, the endoscope may include an elongated portion in which the imaging optical system is arranged at a tip portion thereof and the library data may include a parameter which is determined by a sum of an inclination of a longitudinal axis of the elongated portion and an amount of curvature of the curved part on base coordinates and which is related to an orientation of the imaging optical system on the base coordinates.

According to this configuration, an orientation of the imaging optical system as viewed from the base coordinates can be made consistent. Accordingly, since the orientation of the imaging optical system does not change even if a position of an observed tissue or an inclination of the elongated portion of the endoscope changes according to individual variability, an endoscopic field of view set in advance can be readily and accurately reproduced.

In the endoscope system according to the aspect described above, the endoscope may be an oblique-viewing endoscope which includes a lens barrel unit housing the imaging optical system and in which an optical axis of the imaging optical system is arranged inclined with respect to a central axis of the lens barrel unit, and the field-of-view direction changing unit may change an angle around the central axis of the lens barrel unit.

Changing an angle around the central axis of the lens barrel unit causes a field-of-view direction of the oblique-viewing endoscope with a certain angle with respect to the central axis of the lens barrel unit to change around the central axis of the lens barrel unit and enables the field-of-view direction of the oblique-viewing endoscope to be switched to obliquely upward or obliquely downward. Accordingly, the field-of-view direction of the oblique-viewing endoscope can be oriented toward a desired observation object by simply rotating the lens barrel unit around the central axis. As a result, even when an oblique-viewing endoscope is adopted as the endoscope, an observation object designated by an operator can be readily placed inside the endoscopic field of view.

In the endoscope system according to the aspect described above, the endoscope may be a forward-viewing endoscope which includes a lens barrel unit housing the imaging optical system and in which a central axis of the lens barrel unit and an optical axis of the imaging optical system are arranged so as to match with each other, and the field-of-view direction changing unit may change an angle around a rotational axis which is orthogonal to the central axis of the lens barrel unit in accordance with a change in a position at which the observation object is captured on a display screen displaying image information acquired by the imaging optical system.

Due to an angle around the rotational axis which is orthogonal to the central axis of the lens barrel unit being changed by the field-of-view direction changing unit in accordance with a change in a position at which the observation object is captured on the display screen, the field-of-view direction of the forward-viewing endoscope is changed to a direction which causes the observation object to be captured at a position after the change on the display screen. Accordingly, for example, when the position at which the observation object is captured is changed to an end of the display screen, an endoscopic field of view of an angle of looking into the observation object can be created by changing the field-of-view direction of the forward-viewing endoscope to a direction in which the observation object is captured at the end of an angle of view. As a result, even when a forward-viewing endoscope is adopted as the endoscope, an observation object designated by an operator can be readily placed inside the endoscopic field of view.

In the endoscope system according to the aspect described above, the processor may recognize the observation object by processing image information acquired by the imaging optical system.

According to this configuration, the operator need not specify the observation object from the image information and the operator's effort can be further reduced.

The endoscope system according to the aspect described above may include an input unit which causes a user to designate the observation object based on image information acquired by the imaging optical system.

According to this configuration, the operator can select a desired observation object.

Another aspect of the present disclosure is a procedure support method, including steps of: calling, from a storage apparatus, library data associated with any procedural scene among a plurality of pieces of library data for realizing an endoscopic field of view, each piece of library data being associated with each procedural scene; and changing at least any one of a field-of-view direction, a position, and an attitude of an endoscope for photographing an observation object based on the called library data, wherein each piece of the library data includes at least one relative parameter related to relative positions and attitudes of an imaging optical system of the endoscope and the observation object.

In the procedure support method according to the aspect described above, after specifying the procedural scene by processing image information acquired by the imaging optical system, the library data associated with the specified procedural scene may be called from the storage apparatus.

In the procedure support method according to the aspect described above, the relative parameter may be distance information between the imaging optical system and the observation object, and a distance between the imaging optical system and the observation object as calculated from image information acquired by the imaging optical system may be matched with the distance information by changing at least one of a field-of-view direction, a position, or an attitude of the endoscope.

In the procedure support method according to the aspect described above, the relative parameter may be orientation information of the imaging optical system with respect to the observation object, and an orientation of the imaging optical system with respect to the observation object as calculated from image information acquired by the imaging optical system may be matched with the orientation information by changing at least one of a field-of-view direction, a position, or an attitude of the endoscope.

In the procedure support method according to the aspect described above, the relative parameter may be inclination information around an optical axis of the imaging optical system of the observation object with an elongated shape which extends in a direction orthogonal to the optical axis, and an inclination of the observation object with an elongated shape around the optical axis of the imaging optical system as calculated from image information acquired by the imaging optical system may be matched with the inclination information by changing at least one of a field-of-view direction, a position, or an attitude of the endoscope.

In the procedure support method according to the aspect described above, the endoscope may include a curved part capable of changing an angle of the imaging optical system, and a field-of-view direction of the endoscope may be changed by changing an angle of the imaging optical system using the curved part. In such a case, the endoscope may include an elongated portion in which the imaging optical system is arranged at a tip portion thereof and the library data may include a parameter which is determined by a sum of an inclination of a longitudinal axis of the elongated portion and an amount of curvature of the curved part of the endoscope on base coordinates and which is related to an orientation of the imaging optical system on the base coordinates.

In the procedure support method according to the aspect described above, the endoscope may be an oblique-viewing endoscope which includes a lens barrel unit housing the imaging optical system and in which an optical axis of the imaging optical system is arranged inclined with respect to a central axis of the lens barrel unit, and a field-of-view direction of the endoscope may be changed by changing an angle around the central axis of the lens barrel unit.

In the procedure support method according to the aspect described above, the endoscope may be a forward-viewing endoscope which includes a lens barrel unit housing the imaging optical system and in which a central axis of the lens barrel unit and an optical axis of the imaging optical system are arranged so as to match with each other, and the field-of-view direction of the endoscope may be changed by changing an angle around a rotational axis which is orthogonal to the central axis of the lens barrel unit in accordance with a change in a position at which the observation object is captured on a display screen displaying image information acquired by the imaging optical system.

In the procedure support method according to the aspect described above, the observation object may be recognized by having a processor process image information acquired by the imaging optical system.

In the procedure support method according to the aspect described above, the observation object may be designated by a user based on image information acquired by the imaging optical system.

Another aspect of the present disclosure is a procedure support program causing a computer to execute the steps of: calling, from a storage apparatus, library data associated with any procedural scene among a plurality of pieces of library data for realizing an endoscopic field of view, each piece of library data being associated with each procedural scene; and changing at least any one of a field-of-view direction, a position, and an attitude of an endoscope for photographing an observation object based on the called library data, wherein each piece of the library data includes at least one relative parameter related to relative positions and attitudes of an imaging optical system of the endoscope and the observation object.

The procedure support program according to the aspect described above may cause a computer to execute the steps of: specifying the procedural scene by processing image information acquired by the imaging optical system; and calling the library data associated with the specified procedural scene from the storage apparatus.

In the procedure support program according to the aspect described above, the relative parameter may be distance information between the imaging optical system and the observation object, and a distance between the imaging optical system and the observation object as calculated from image information acquired by the imaging optical system may be matched with the distance information by changing at least one of a field-of-view direction, a position, or an attitude of the endoscope.

In the procedure support program according to the aspect described above, the relative parameter may be orientation information of the imaging optical system with respect to the observation object, and an orientation of the imaging optical system with respect to the observation object as calculated from image information acquired by the imaging optical system may be matched with the orientation information by controlling at least one of a field-of-view direction, a position, or an attitude of the endoscope.

In the procedure support program according to the aspect described above, the relative parameter may be inclination information around an optical axis of the imaging optical system of the observation object with an elongated shape which extends in a direction orthogonal to the optical axis, and an inclination of the observation object with an elongated shape around the optical axis of the imaging optical system as calculated from image information acquired by the imaging optical system may be matched with the inclination information by changing at least one of a field-of-view direction, a position, or an attitude of the endoscope.

In the procedure support program according to the aspect described above, the endoscope may include a curved part capable of changing an angle of the imaging optical system, and a field-of-view direction of the endoscope may be changed by changing an angle of the imaging optical system using the curved part. In such a case, the endoscope may include an elongated portion in which the imaging optical system is arranged at a tip portion thereof and the library data may include a parameter which is determined by a sum of an inclination of a longitudinal axis of the elongated portion and an amount of curvature of the curved part on base coordinates and which is related to an orientation of the imaging optical system on the base coordinates.

In the procedure support program according to the aspect described above, the endoscope may be an oblique-viewing endoscope which includes a lens barrel unit housing the imaging optical system and in which an optical axis of the imaging optical system is arranged inclined with respect to a central axis of the lens barrel unit, and a field-of-view direction of the endoscope may be changed by changing an angle around the central axis of the lens barrel unit.

In the procedure support program according to the aspect described above, the endoscope may be a forward-viewing endoscope which includes a lens barrel unit housing the imaging optical system and in which a central axis of the lens barrel unit and an optical axis of the imaging optical system are arranged so as to match with each other, and the field-of-view direction of the endoscope may be changed by changing an angle around a rotational axis which is orthogonal to the central axis of the lens barrel unit in accordance with a change in a position at which the observation object is captured on a display screen displaying image information acquired by the imaging optical system.

The procedure support program according to the aspect described above may cause a computer to execute the step of recognizing the observation object by processing image information acquired by the imaging optical system.

In the procedure support program according to the aspect described above, the observation object may be designated by a user based on image information acquired by the imaging optical system.

According to the present disclosure, by providing an operator with a visual field of observation in accordance with a treatment step and an observation object, an advantageous effect of reducing the operator's effort and enabling treatment to be performed smoothly can be produced.

### REFERENCE SIGNS LIST

1 endoscope system
3 electric scope (endoscope)
5 robot arm (electric arm, field-of-view direction changing unit)
13 insertion portion (elongated portion)
17 curved part
21 main controller (processor)
23 curvature controller (processor)
25 position/attitude controller (processor)
27 auxiliary storage apparatus (storage apparatus)
35 headset (input unit)
37 hand switch (input unit)
39 foot switch (input unit)
41 oblique-viewing endoscope (endoscope)
43 lens barrel unit
47 electric attachment (field-of-view direction changing unit)
51 forward-viewing endoscope (endoscope)
53 lens barrel unit
S observation object

1. An endoscope system comprising:
an endoscope that comprises an imaging optical system for photographing an observation object;
an electric arm that changes a position and an attitude of the endoscope while supporting the endoscope;
a field-of-view direction changing unit capable of changing a field-of-view direction of the endoscope;
a storage apparatus that stores a plurality of pieces of library data for realizing an endoscopic field of view, each piece of library data being associated with each procedural scene; and
at least one processor comprising hardware,
wherein:
the endoscope comprises an elongated portion in which the imaging optical system is arranged at a tip portion thereof,
each piece of library data comprises at least one relative parameter related to a relative position and a relative attitude between the imaging optical system and the observation object,
the relative parameter comprises at least one of a first parameter or a second parameter, the first parameter being determined by a sum of an inclination of a longitudinal axis of the elongated portion and an amount of curvature of the field-of-view direction changing unit on base coordinates, the first parameter being related to an orientation of the imaging optical system on the base coordinates, the second parameter indicating an orientation of the imaging optical system as viewed from a coordinate of the observation object, and
the processor is configured to call the library data associated with any procedural scene from the storage apparatus, and subsequently control at least one of the field-of-view direction changing unit or the electric arm based on the called library data.

2. The endoscope system according to claim 1, wherein the processor is configured to specify the procedural scene by processing image information acquired by the imaging optical system, and subsequently call the library data associated with the specified procedural scene from the storage apparatus.

3. The endoscope system according to claim 1, wherein:
the relative parameter is distance information between the imaging optical system and the observation object, and
the processor is configured to match a distance between the imaging optical system and the observation object as calculated from image information acquired by the imaging optical system with the distance information by controlling at least one of the field-of-view direction changing unit or the electric arm.

4. The endoscope system according to claim 1, wherein:
the relative parameter is orientation information of the imaging optical system with respect to the observation object, and
the processor is configured to match the orientation of the imaging optical system with respect to the observation object as calculated from image information acquired by the imaging optical system with the orientation information by controlling at least one of the field-of-view direction changing unit and the electric arm.

5. The endoscope system according to claim 1, wherein:
the relative parameter is inclination information around an optical axis of the imaging optical system of the observation object with an elongated shape which extends in a direction orthogonal to the optical axis, and
the processor is configured to match an inclination of the observation object with an elongated shape around the optical axis of the imaging optical system as calculated from image information acquired by the imaging optical system with the inclination information by controlling at least one of the field-of-view direction changing unit or the electric arm.

6. The endoscope system according to claim 1, wherein the field-of-view direction changing unit is a curved part capable of changing an angle of the imaging optical system in the endoscope.

7. The endoscope system according to claim 1, wherein:
the endoscope is an oblique-viewing endoscope that has a lens barrel unit housing the imaging optical system and in which an optical axis of the imaging optical system is arranged inclined with respect to a central axis of the lens barrel unit, and
the field-of-view direction changing unit is configured to change an angle around the central axis of the lens barrel unit.

8. The endoscope system according to claim 1, wherein:
the endoscope is a forward-viewing endoscope that has a lens barrel unit housing the imaging optical system and in which a central axis of the lens barrel unit and an optical axis of the imaging optical system are arranged so as to match with each other, and
the field-of-view direction changing unit is configured to change an angle around a rotational axis orthogonal to the central axis of the lens barrel unit in accordance with a change in a position at which the observation object is captured on a display screen displaying image information acquired by the imaging optical system.

9. The endoscope system according to claim 1, wherein the processor is configured to recognize the observation object by processing image information acquired by the imaging optical system.

10. The endoscope system according to claim 1, further comprising an input unit that causes a user to designate the observation object based on image information acquired by the imaging optical system.

11. A procedure support method comprising steps of:
calling, from a storage apparatus, library data associated with any procedural scene among a plurality of pieces of library data for realizing an endoscopic field of view, each piece of library data being associated with each procedural scene; and
changing at least any one of a field-of-view direction, a position, or an attitude of an endoscope for photographing an observation object based on the called library data,
wherein:
the endoscope comprises an elongated portion in which the imaging optical system is arranged at a tip portion thereof,
each piece of the library data comprises at least one relative parameter related to a relative position and a relative attitude between an imaging optical system of the endoscope and the observation object, and
the relative parameter comprises at least one of a first parameter or a second parameter, the first parameter being determined by a sum of an inclination of a longitudinal axis of the elongated portion and an amount of curvature of the field-of-view direction changing unit on base coordinates, the first parameter being related to an orientation of the imaging optical system on the base coordinates, the second parameter indicating the orientation of the imaging optical system as viewed from a coordinate of the observation object.

12. The procedure support method according to claim 11, wherein the procedural scene is specified by processing image information acquired by the imaging optical system, and subsequently the library data associated with the specified procedural scene is called from the storage apparatus.

13. The procedure support method according to claim 11, wherein:
the relative parameter is distance information between the imaging optical system and the observation object, and
a distance between the imaging optical system and the observation object as calculated from image information acquired by the imaging optical system is matched with the distance information by changing at least one of a field-of-view direction, a position, or an attitude of the endoscope.

14. The procedure support method according to claim 11, wherein:
the relative parameter is orientation information of the imaging optical system with respect to the observation object, and
an orientation of the imaging optical system with respect to the observation object as calculated from image information acquired by the imaging optical system is matched with the orientation information by changing at least one of a field-of-view direction, a position, and an attitude of the endoscope.

15. The procedure support method according to claim 11, wherein:
the endoscope comprises a curved part capable of changing an angle of the imaging optical system, and
a field-of-view direction of the endoscope is changed by changing an angle of the imaging optical system by means of the curved part.

16. The procedure support method according to claim 11, wherein:
the endoscope is an oblique-viewing endoscope that has a lens barrel unit housing the imaging optical system and in which an optical axis of the imaging optical system is arranged inclined with respect to a central axis of the lens barrel unit, and
a field-of-view direction of the endoscope is changed by changing an angle around the central axis of the lens barrel unit.

17. The procedure support method according to claim 11, wherein:
the endoscope is a forward-viewing endoscope that has a lens barrel unit housing the imaging optical system and in which a central axis of the lens barrel unit and an optical axis of the imaging optical system are arranged so as to match with each other, and
the field-of-view direction of the endoscope is changed by changing an angle around a rotational axis orthogonal to the central axis of the lens barrel unit in accordance with a change in a position at which the observation object is captured on a display screen displaying image information acquired by the imaging optical system.

18. The procedure support method according to claim 11, wherein the observation object is recognized by a processor processing image information acquired by the imaging optical system.

19. The procedure support method according to claim 11, wherein the observation object is designated by a user based on image information acquired by the imaging optical system.

20. A non-transitory computer-readable recording medium storing a procedure support program causing a computer to execute steps of:
calling, from a storage apparatus, library data associated with any procedural scene among a plurality of pieces of library data for realizing an endoscopic field of view, each piece of library data being associated with each procedural scene; and
changing at least any one of a field-of-view direction, a position, or an attitude of an endoscope for photographing an observation object based on the called library data,
wherein:
the endoscope comprises an elongated portion in which the imaging optical system is arranged at a tip portion thereof,
each piece of the library data comprises at least one relative parameter related to a relative position and a relative attitude between an imaging optical system of the endoscope and the observation object, and
the relative parameter comprises at least one of a first parameter or a second parameter, the first parameter being determined by a sum of an inclination of a longitudinal axis of the elongated portion and an amount of curvature of the field-of-view direction changing unit on base coordinates, the first parameter being related to an orientation of the imaging optical system on the base coordinates, the second parameter indicating the orientation of the imaging optical system as viewed from a coordinate of the observation object.
